(19) United States
(12) Patent Application Publication
(10) Pub. No.: US 2007/0250008 A1
(43) Pub. Date: Oct. 25, 2007

(54) Fluid injection apparatus and adaptor pump therefor

(76) Inventor: Eugene A. Gelblum, Pittsburgh, PA (US)

Correspondence Address: GREGORY L BRADLEY;MEDRAD INC, ONE MEDRAD DRIVE, INDIANOLA, PA, 15051, US

(21) Appl. No.: 11/407,804

(22) Filed: Apr. 20, 2006

(51) Int. Cl. A61M 1/00

(52) U.S. Cl. 604/151

(57) ABSTRACT

The fluid injection apparatus includes an injector having an injector head and a drive piston extendable from the injector head. A pressure jacket is engaged with the injector head and defines a receiving opening. An adaptor pump is inserted into the receiving bore in the pressure jacket. The adaptor pump includes a pressurizing body in which a pressurizing plunger is seated. The pressurizing body is in fluid communication with a container body defining an internal chamber which is pressurized by the action of the plunger in the pressurizing body. The container body defines an opening allowing access to the internal or pressurizing chamber for accepting a pinch sleeve into the container body. The pinch sleeve is inserted into the container body through the opening and is disposed in the pressurizing chamber. The pinch sleeve is adapted to receive a flexible or collapsible pouch containing medical liquid.

### BACKGROUND OF THE INVENTION

1. Field of the Invention

The present invention relates to the injection of medical liquids into patients and, more specifically, relates to a fluid injection apparatus adapted to dispense a medical liquid contained in a flexible pouch or bag by applying external pressure to the pouch and an adaptor pump for use in such a fluid injection apparatus.

2. Description of Related Art

In many medical diagnostic and therapeutic procedures, a medical practitioner such as a physician injects a patient with a fluid. In many of these procedures, the injection device is a syringe that contains the medical liquid to be injected into the patient. The syringe has an injection tip which is connected to a hypodermic or intravenous needle cannula or to a catheter by interconnecting flexible tubing. In recent years, a number of injector-actuated syringes and powered injectors for pressurized injection of fluids, such as contrast media, have been developed for use in procedures such as angiography, computed tomography (“CT”), ultrasound, and NMR/MRI. In general, these powered injectors are designed to deliver a preset amount of contrast media at a preset flow rate through the use of a removable and disposable syringe.

Angiography is used in the detection and treatment of abnormalities or restrictions in blood vessels. In an angiographic procedure, a radiographic image of a vascular structure is obtained through the use of radiographic contrast medium, sometimes referred to simply as contrast, which is injected through a catheter. The vascular structures in which the contrast is injected are filled with contrast. X-rays are passed through the region of interest and are absorbed by the contrast, causing a radiographic outline or image of the blood vessels containing the contrast. The resulting images may be displayed on, for example, a video monitor and recorded.

In a typical angiographic procedure, the medical practitioner places a cardiac catheter into a vein or artery. The catheter is connected to either a manual or to an automatic contrast injection mechanism. A typical manual contrast injection mechanism includes a syringe in fluid connection with the catheter. The fluid path also includes, for example, a source of contrast, a source of flushing fluid, typically saline, and a pressure transducer to measure patient blood pressure. In a typical system, the source of contrast is connected to the fluid path via a valve, for example, a three-way stopcock. The source of saline and the pressure transducer may also be connected to the fluid path via additional valves. The operator of the manual system controls the syringe and each of the valves to draw saline or contrast into the syringe and to inject the contrast or saline into the patient through the catheter. The operator of the syringe may adjust the flow rate and volume of injection by altering the force applied to the plunger of the syringe. Manual sources of fluid pressure and flow used in these medical applications, such as syringes and manifolds, therefore require operator effort which provides feedback of the fluid pressure/flow generated to the operator. The feedback is desirable but the operator effort often leads to fatigue. Thus, fluid pressure and flow may vary depending on the operator's strength and technique.

Automatic contrast injection mechanisms typically include a syringe connected to a powered injector having, for example, a powered linear actuator. Typically, an operator enters settings into an electronic control system of the powered injector, for example, for a fixed volume of contrast and a fixed rate of injection. In many systems, there is no interactive control between the operator and the powered injector except to start or stop the injection. A change in flow rate in such systems occurs simply by stopping the machine and resetting the injection parameters. Automation of angiographic procedures using powered injectors is discussed, for example, in U.S. Pat. Nos. 5,460,609; 5,573,515; and 5,800,397.

Conventional syringes are often prepared by filling and air removal from flasks of different contents. In some instances, syringes are prefilled by the manufacturer and sealed in a sterile state for use by a medical practitioner in a fluid injection procedure. In addition to syringes, medical fluids are often stored in packaging in the form of a flexible and collapsible pouch or bag fitted with a coupling element. The pouch replaces the traditional syringe. The pouch coupling is connected to flexible tubing which is linked to an injection conduit, such as a catheter, intravenous needle, or other such structure. It is generally known to place such a filled, flexible pouch in a casing or container that includes an inert motive liquid which is placed under pressure, such as the apparatus described in U.S. Pat. No. 3,199,511 to Kulick. The apparatus disclosed in U.S. Pat. No. 3,199,511 includes a collapsible pouch or bag which contains a medical fluid to be administered to a patient. The collapsible bag is immersed in a container of incompressible fluid which completely surrounds the bag. Additional incompressible fluid is then pumped into the container. As additional incompressible fluid is pumped into the container at a regulated state, the medical fluid is expelled from the bag at the same rate for administration of the medical liquid to the patient. This apparatus is specifically used for the administration of parenteral fluids, blood, and chemotherapy agents in definite amounts over a period of time.

Another example of a device adapted to administer a medical liquid contained in a flexible pouch or bag is disclosed in U.S. Pat. No. 6,056,724 to Lacroix. The device disclosed in this patent includes a flexible pouch or bag that contains the medical liquid to be injected into a patient. The pouch has an outlet opening for connection to flexible tubing linked to an injection conduit, such as a catheter, intravenous needle, or other such structure. The flexible pouch is deformed by an inert motive liquid which is placed under pressure in a casing containing the motive liquid. The motive liquid transmits pressure to the flexible pouch forcing the contents of the flexible pouch into the connecting flexible tubing.

In general, injection mechanisms adapted to accept and actuate syringes and the foregoing devices adapted to administer a medical liquid contained in a flexible pouch or bag are mutually exclusive platforms for administering a medical fluid to a patient. In other words, syringe injectors cannot accept filled fluid-filled pouches or bags and the devices disclosed, for example, by Kulick and Lacroix are not adapted to accept syringes. As a result, hospitals and other medical facilities must maintain separate pieces of equipment for dispensing medical fluid from both types of fluid-containing bodies. In the case of syringes, a syringe injector with an injector head is required. In the case of flexible pouches, an enclosure adapted to contain the motive liquid and an associated pressurizing pump are required. In many therapeutic settings, such as the cardiac suite of a hospital, equipment space is at a premium and it would be advantageous if a single piece of equipment could be provided which is adapted to accept and actuate syringes and dispense fluid from fluid-filled flexible pouches or bags.

### SUMMARY OF THE INVENTION

One aspect of the invention relates to an adaptor pump for use in a fluid injection apparatus. The adaptor pump comprises a pressurizing body and a plunger seated in the pressurizing body and a container body defining an internal chamber which is in fluid communication with the interior of the pressurizing body. The container body defines an opening allowing access to the internal chamber. A pinch sleeve is inserted in the container body opening and is disposed in the internal chamber. The pinch sleeve is adapted to receive a flexible pouch containing medical liquid. A closure closes the container body opening and encloses the pinch sleeve in the internal chamber. A pouch sensor may be disposed in the pinch sleeve.

The pressurizing body and container body may be integrally formed of molded plastic material. In one variation, the pressurizing body and container body may be connected by a bridge. The interior of the pressurizing body and the internal chamber defined by the container body may be in fluid communication via a port defined in the bridge.

The pinch sleeve may be in the form of an elastomeric sleeve shaped to receive the flexible pouch. The closure defines an aperture for passage of medical tubing for connection to a discharge port or coupling on the flexible pouch.

The pressurizing body may comprise an expansion section at a proximal end of the pressurizing body which is adapted to expand radially when the plunger is disposed in the expansion section. Additionally, a wall thickness of the pressurizing body may narrow to a reduced wall thickness at the expansion section such that an inner diameter of the expansion section is larger than the inner diameter of the pressurizing body. In another variation, an outer surface of the pressurizing body may be tapered or stepped inward toward a central axis of the pressurizing body and/or an inner surface of the pressurizing body may be tapered or stepped outward away from the central axis of the pressurizing body to form the reduced wall thickness.

Another aspect of the invention relates to fluid injection apparatus that generally comprises a pressurizing device adapted for fluid connection to an incompressible fluid source and a container body which defines an internal chamber in fluid communication with the pressurizing device. The container body defines an opening allowing access to the internal chamber. A pinch sleeve is inserted in the container body opening and is disposed in the internal chamber. The pinch sleeve is adapted to receive a flexible pouch containing medical liquid. A closure closes the container body opening to enclose the pinch sleeve in the internal chamber. The pressurizing device is adapted to pump incompressible fluid into the internal chamber such that increasing hydraulic pressure in the internal chamber squeezes the pinch sleeve to expel medical fluid from the flexible pouch through a discharge port.

The pinch sleeve may be in the form of an elastomeric sleeve shaped to receive the flexible pouch. The closure defines an aperture for passage of medical tubing for connection to the discharge port on the flexible pouch.

In another embodiment, the fluid injection apparatus comprises an injector comprising an injector head and a drive piston extendable from the injector head, and an adaptor pump associated with the injector head and comprising a pressurizing body and a plunger seated in the pressurizing body. A container body is in fluid communication with the interior of the pressurizing body and defines an internal chamber in fluid communication with the interior of the pressurizing body. The container body defines an opening allowing access to the internal chamber. A pinch sleeve is inserted in the container body opening and is disposed in the internal chamber. The pinch sleeve is adapted to receive a flexible pouch containing medical liquid. A closure closes the container body opening to enclose the pinch sleeve in the internal chamber.

The pressurizing body and container body may be integrally formed of molded plastic material. In one variation, the pressurizing body and container body may be connected by a bridge. The interior of the pressurizing body and internal chamber may be in fluid communication via a port defined in the bridge.

A pressure jacket may be engaged with the injector head. The pressure jacket defines a receiving bore for receiving the pressurizing body to associate the pressurizing body with the injector head. In one variation, the pressure jacket may define a longitudinal slot for receiving the bridge connecting the pressurizing body and the container body. At least one light source may be associated with the injector head and be positioned to illuminate at least the pressurizing body received in the pressure jacket.

The pinch sleeve may be in the form of an elastomeric sleeve shaped to receive the flexible pouch. The closure defines an aperture for passage of medical tubing for connection to a coupling on the flexible pouch.

The pressurizing body may comprise an expansion section at a proximal end of the pressurizing body which is adapted to expand radially when the plunger is disposed in the expansion section. Additionally, a wall thickness of the pressurizing body may narrow to a reduced wall thickness at the expansion section such that an inner diameter of the expansion section is larger than the inner diameter of the pressurizing body. In another variation, an outer surface of the pressurizing body may be tapered or stepped inward toward a central axis of the pressurizing body and/or an inner surface of the pressurizing body may be tapered or stepped outward away from the central axis of the pressurizing body to form the reduced wall thickness.

The injector may further comprise one or more support arms associated with and extending outward from the injector head and a retaining member associated with the at least one support arm. The retaining member defines a receiving slot for receiving a distal section of the pressurizing body. The support arm or arms may be movable between a first position wherein the retaining member prevents removal of the pressurizing body from a pressure jacket supporting the pressurizing body and a second position wherein the pressurizing body is removable from the pressure jacket.

In another aspect, the invention relates to a method of preparing a fluid injection apparatus for a fluid injection procedure. The method comprises providing an injector comprising an injector head and a drive piston extendable from the injector head, providing an adaptor pump comprising a pressurizing body and container body in mutual fluid communication, associating the pressurizing body with the injector head, inserting a pinch sleeve into a container body, and inserting a flexible pouch containing medical liquid into the pinch sleeve. The adaptor pump comprises a pressurizing body having a plunger seated therein and the drive piston associated with the injector head is adapted to impart motive forces to the plunger. The container body defines an internal chamber which is in fluid communication with the interior of the pressurizing body and defines an opening allowing access to the internal chamber. The pinch sleeve is inserted into the container body through the container body opening and the pinch sleeve is adapted to receive the flexible pouch containing medical liquid.

The method may additionally comprise closing the container body opening with a closure to enclose the pinch sleeve in the internal chamber and connecting medical tubing to a discharge port or coupling on the flexible pouch through an aperture defined in the closure.

The step of associating the pressurizing body with the injector head may comprise inserting the pressurizing body into a pressure jacket connected to the injector head. Further, the method may further comprise detecting at least the presence of the pouch in the pinch sleeve with a pouch sensor disposed in the pinch sleeve.

Further details and advantages of the invention will become clear upon reading the following detailed description in conjunction with the accompanying drawing figures, wherein like parts are identified with like reference numerals throughout

### BRIEF DESCRIPTION OF THE DRAWINGS

FIG. 1 is an exploded perspective view of an adaptor pump pursuant to an embodiment of the invention.

FIG. 2 is an assembled perspective view of the adaptor pump of FIG. 1.

FIG. 3 is a longitudinal cross-sectional view of the adaptor pump of FIG. 1.

FIG. 4 is a rear perspective view of a plunger used in the adaptor pump of FIG. 1.

FIG. 5 is an exploded perspective of a fluid injection apparatus pursuant to an embodiment of the invention and adapted to accept the adaptor pump of FIG. 1.

FIG. 6 is a longitudinal cross-sectional view of the fluid injection apparatus of FIG. 5 showing the adaptor pump associated with the fluid injection apparatus.

FIG. 7 is a longitudinal cross-sectional view of the fluid injection apparatus of FIG. 5 showing the adaptor pump associated with the fluid injection apparatus and a support structure of the apparatus in a pivoted position allowing insertion and removal of the adaptor pump from the apparatus.

FIG. 8 is frontal perspective view of the fluid injection apparatus of FIG. 5 showing the adaptor pump associated therewith and showing an injector head of the apparatus.

FIG. 9 is an exploded perspective view of the fluid injection apparatus of FIG. 8 showing further features of the injector head.

FIG. 10 is a perspective view of another embodiment of the fluid injection apparatus.

### DESCRIPTION OF THE PREFERRED EMBODIMENTS

For purposes of the description hereinafter, spatial orientation terms, if used, shall relate to an embodiment of the invention as it is oriented in the accompanying drawing figures or otherwise described in the following description of the invention. However, it is to be understood that the invention embodiments described hereinafter may assume many alternative variations and configurations. It is also to be understood that the specific devices illustrated in the accompanying drawing figures and described herein are simply exemplary to the invention and should not be considered as limiting.

The invention described herein, in one embodiment, relates to a fluid injection apparatus suitable for accepting and actuating a device adapted to deliver medical fluid(s) to a patient. In one embodiment, the apparatus is adapted to accept and actuate a pump device which is formed with a chamber sized and configured to accommodate a fluid-filled flexible pouch or bag, such and a fluid-filled flexible pouch that is connected to a patient via an intravenous (“IV”) needle cannula or catheter for delivering the contents of the pouch intravenously to the patient. Such an IV pouch may contain liquid drugs, for example, chemotherapy drugs, contrast media for use in computed tomography (“CT”) or angiography procedures, or other medical fluids which are routinely delivered to patients in medical settings such as saline and glucose solutions. In any of the foregoing situations, the fluid injection apparatus described herein is capable of positively controlling the total quantity of fluid delivered and the flow rate of delivery. In one particular form, the fluid injection apparatus includes an injector with an injector head and support structure adapted to support an “adaptor” pump which interfaces with the injector head. The adaptor pump comprises a pressurizing body in fluid communication with a container body which defines an internal chamber adapted to accept a fluid-filled IV pouch of bag. The container body defines an opening allowing access to the internal chamber and the loading of a fluid-filled pouch in the internal chamber. In use, once a fluid-filled pouch or bag is loaded into the internal chamber, fluid pressure is applied to the pouch to dispense the contents of the pouch. The adaptor pump is adapted to pressurize the internal chamber with an inert, motive liquid which hydraulically compresses the fluid-filled pouch and thereby expels the contents of the pouch. The pouch is connected via suitable medical tubing and connectors to a fluid delivery device such as an IV needle cannula or a catheter. The adaptor pump may be operated to dispense the pouch contents at a controlled flow rate.

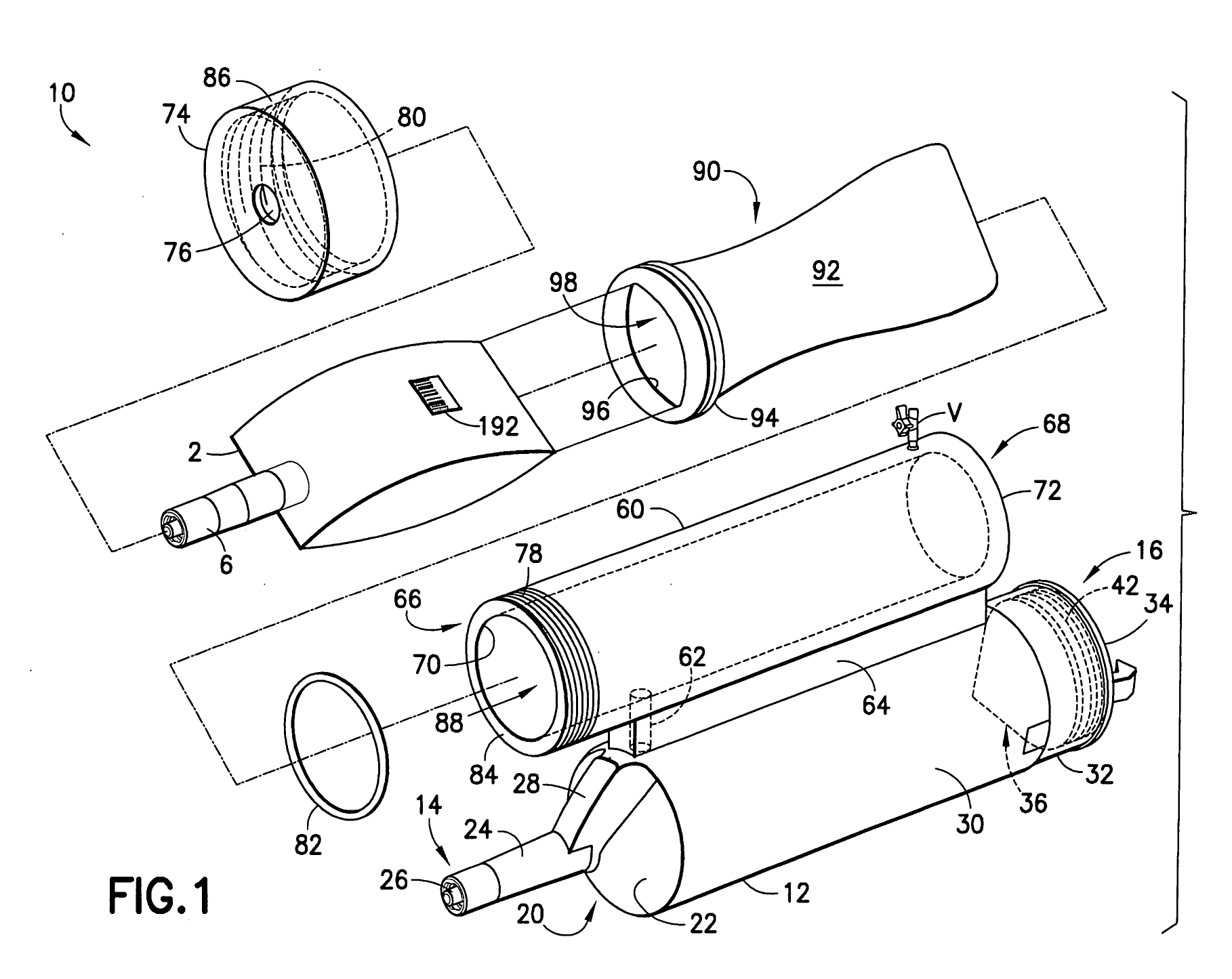

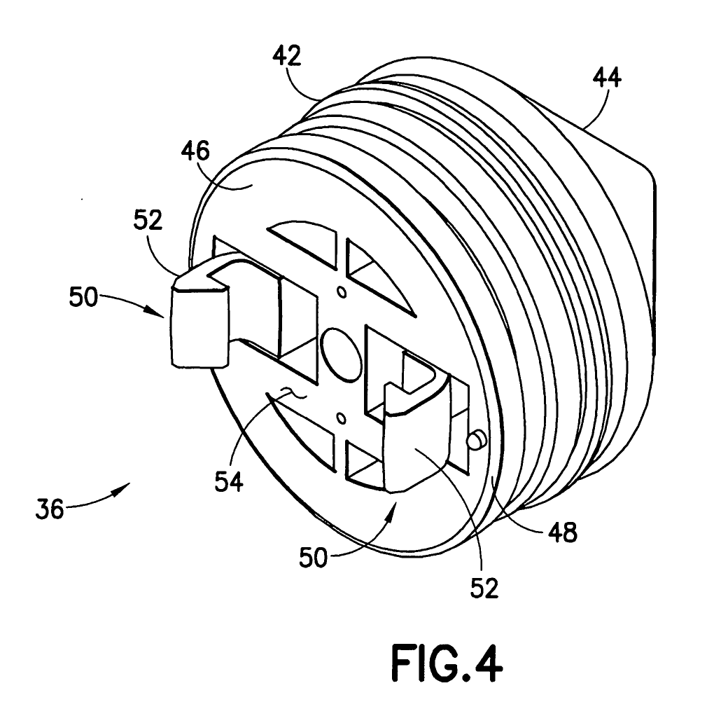

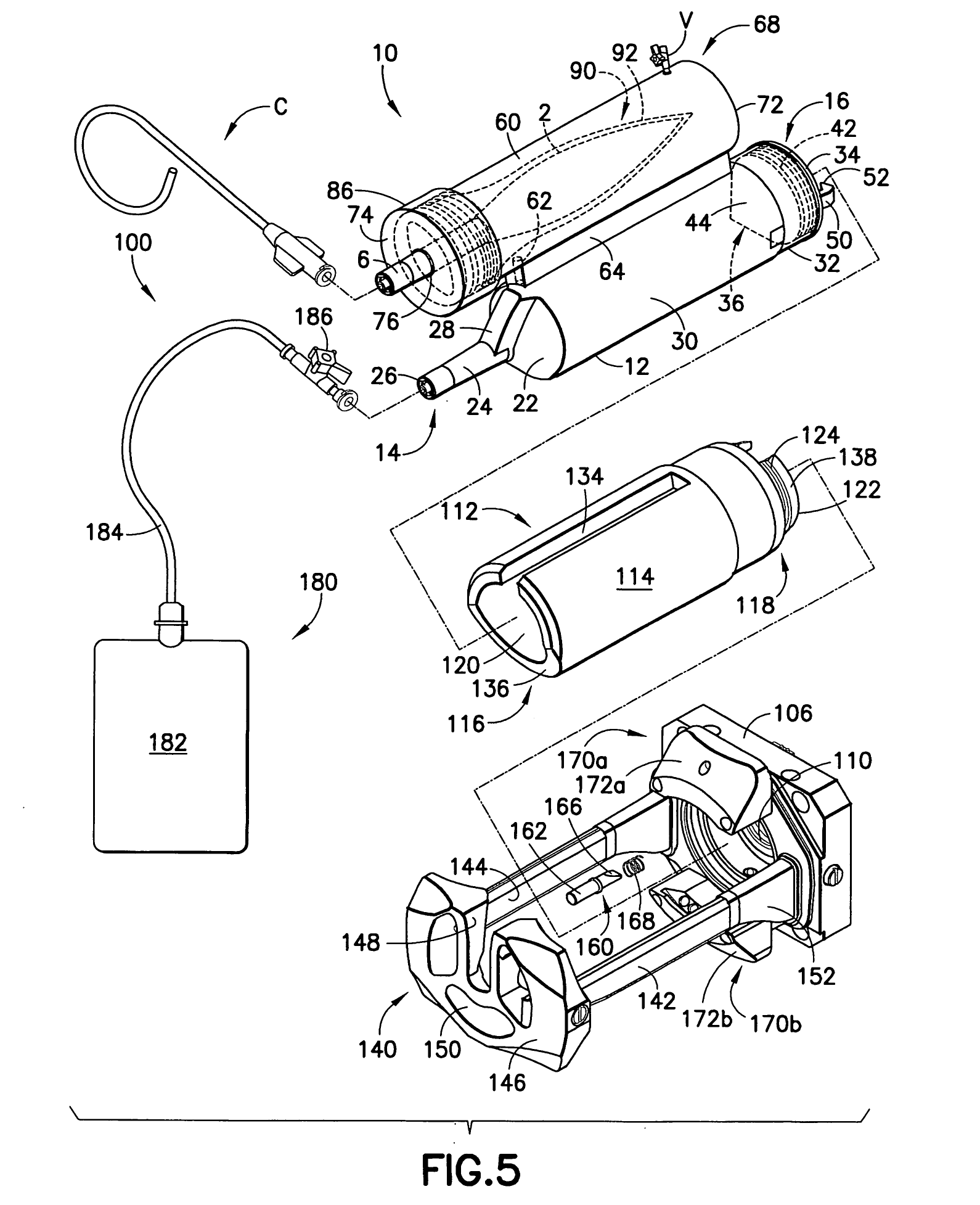

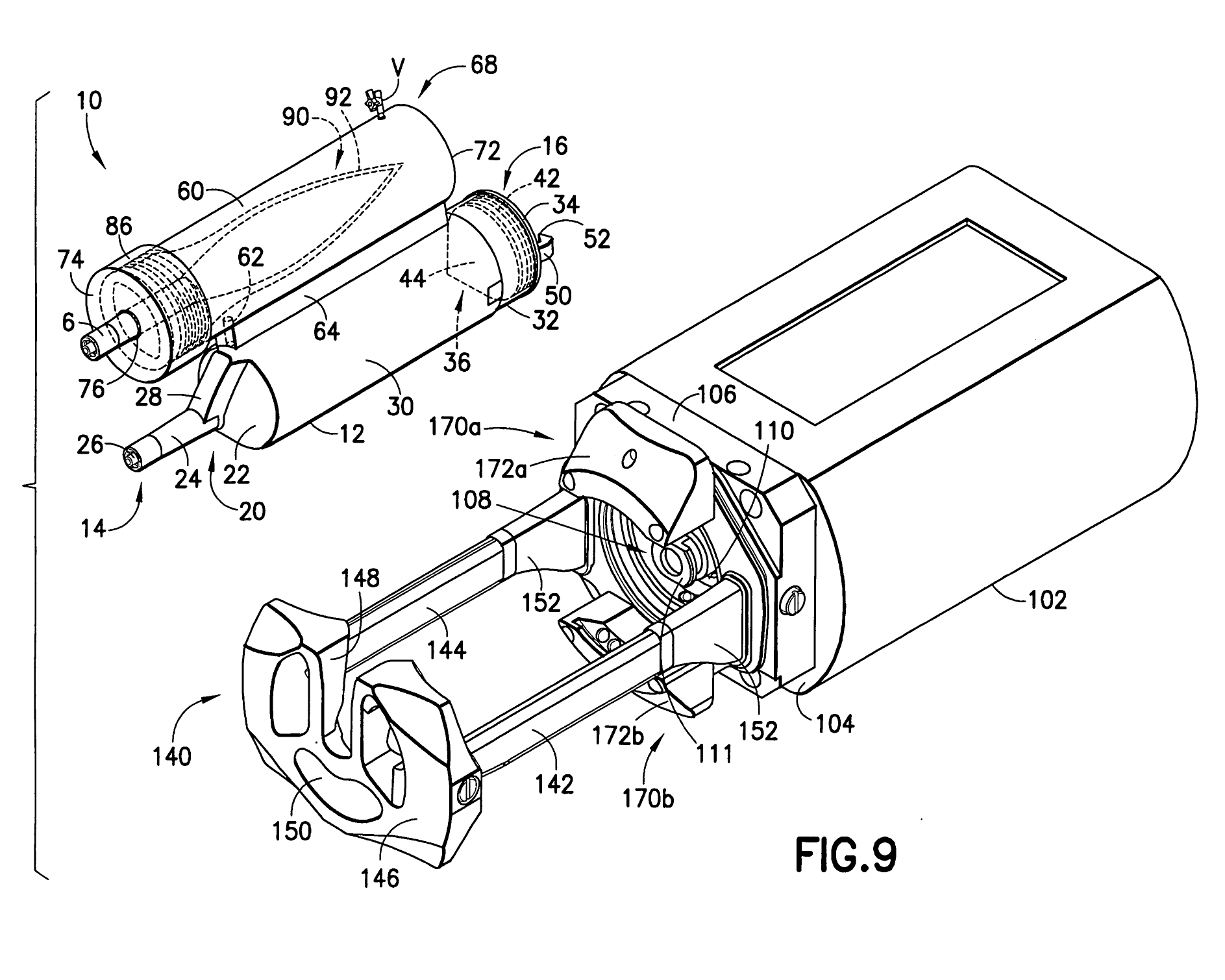

FIGS. 1-4 show an embodiment of the invention. FIGS. 1-4 show an adaptor pump 10 for use or association with a fluid injection apparatus 100 discussed herein in connection with FIG. 5-9. Adaptor pump 10 generally comprises an elongated and generally cylindrical syringe-shaped body 12 having a front or distal end 14 and a rear or proximal end 16. Body 12 is generally adapted to pressurizing a second body with a pressurizing hydraulic fluid, as discussed fully herein, and will be referred to hereinafter as a “pressurizing” body 12 to reflect this general function. Thus, pressurizing body 12, which is shown in the general form of a syringe body, reflects one desirable form of a pressurizing device in accordance with the present invention for pressurizing a second body with pressurizing hydraulic fluid. However, this terminology is not intended to limit the scope of the invention as claimed in the appended claims. Pressurizing body 12 may take other forms such as a positive displacement pump or a sliding piston pump driven by a controlled electric motor, or other similar devices.

Pressurizing body 12 has a body wall 18. Pressurizing body 12 defines a distally-extending section 20 adapted to support adaptor pump 10 in fluid injection apparatus 100 and interact with supporting structure associated with fluid injection apparatus 100 as discussed herein. Distal section 20 is formed at the distal end 14 of pressurizing body 12 and is further adapted to permit the filling of pressurizing body 12 with an inert, motive “pressurizing” liquid as discussed herein. Distal section 20 includes a generally conical-shaped portion 22 which tapers inward toward a central axis L₁ of pressurizing body 12 and an elongated neck 24 which tapers from conical portion 22 and terminates in a distal end connecting structure 26, typically a luer connecting structure or connector. Luer connector 26 is adapted to engage a mating luer connector for associating adaptor pump 10 with a supply of inert, motive liquid which is used as pressurizing hydraulic fluid as discussed herein and, further, for connecting pressurizing body 12 to a fluid waste container as necessary as also discussed herein. Pressurizing body 12 is desirably formed of molded plastic material of a type customary in the medical field for the production of syringes for medical applications. However, pressurizing body 12 may also be made of non-plastic materials such as metal, for example, aluminum.

Conical portion 22 defines a raised or projecting alignment flange or tab member 28. Alignment flange 28 defines a hollow space or area therein and enables viewing of fluid within pressurizing body 12. Additionally, alignment flange 28 acts as a visual indicator for properly aligning adaptor pump 10 for proper operative association with fluid injection apparatus 100. Alignment flange 28 generally extends upward from elongated neck 24 to a center or main body section generally forming a barrel section or portion 30 of pressurizing body 12. Barrel 30 is generally cylindrical in shape and extends from conical portion 22 to an expansion section 32 formed at the proximal end 16 of pressurizing body 12. Expansion section 32 is formed as part of barrel 30, and barrel 30 generally connects distal section 20 to expansion section 32. The function and use of expansion section 32 are discussed further herein. As indicated previously, conical portion 22 of pressurizing body 12 tapers to form elongated neck 24 which has a relatively small inner diameter compared to the inner diameter of barrel 30. At the proximal end 16 of pressurizing body 12, the pressurizing body 12 further includes an outward extending lip or flange 34 which is adapted to engage corresponding structure associated with fluid injection apparatus 100 to, for example, alert the fluid injection apparatus 100 of the presence of adaptor pump 10 and prevent over-insertion of pressurizing body 12 into a receiving pressure jacket associated with fluid injection apparatus 100.

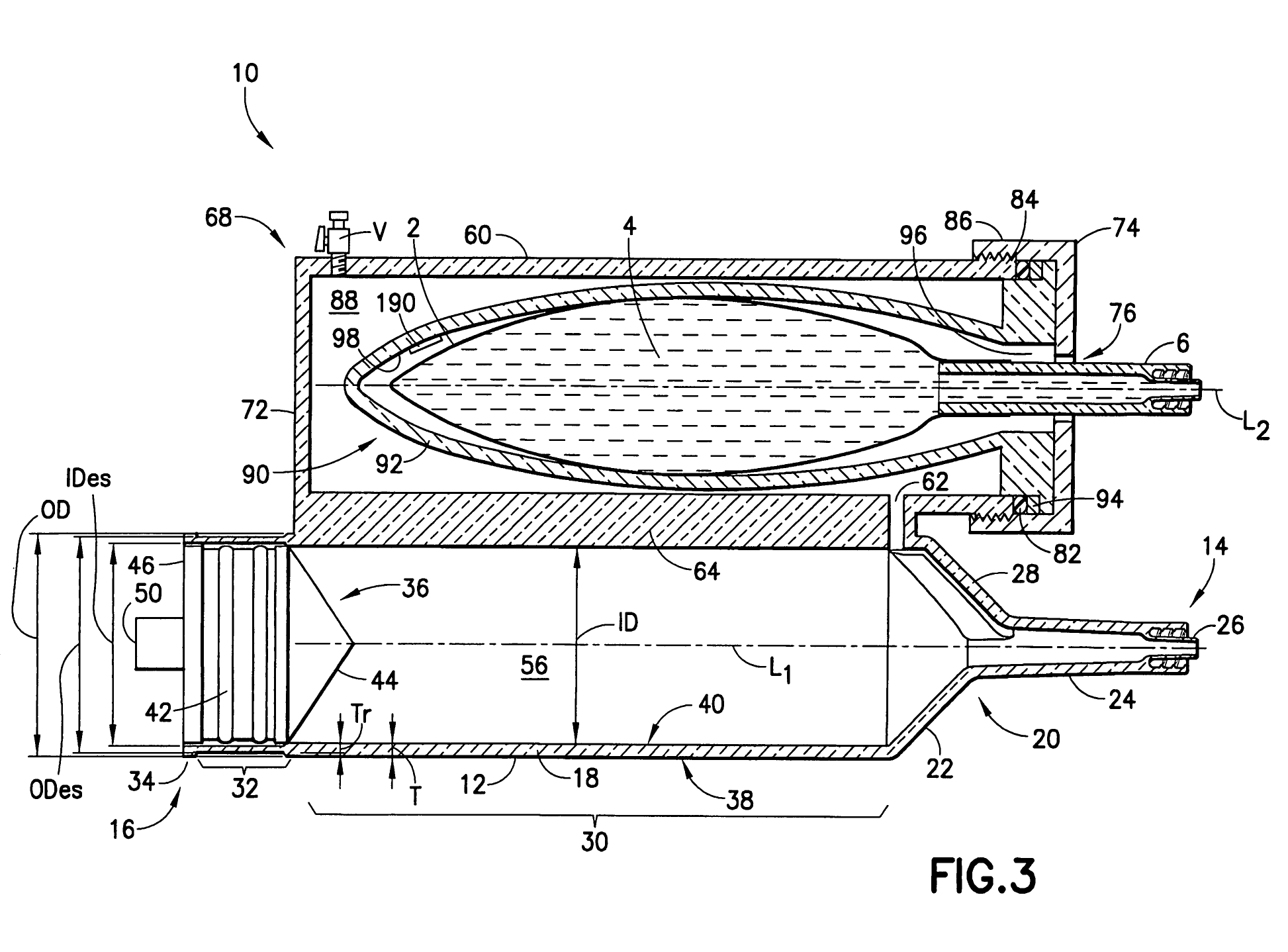

A difficulty with current disposable plastic syringes known in the art for use in fluid injection apparatus such as fluid injection apparatus 100 discussed herein is that such plastic syringes exhibit plastic creep over time and especially during sterilization heat cycles. This causes such plastic syringe to swell, particularly in the area where the syringe plunger is seated. This often makes it difficult to load such prior art plastic syringes in front-loading pressure jackets often associated with fluid injection apparatuses because of induced swelling caused by the syringe plunger. In adaptor pump 10 this disadvantage is overcome by seating and storing a pressurizing plunger 36 associated with adaptor pump 10 in expansion section 32 of barrel 30. Thus, plunger 36 is desirably seated in the expansion section 32 of barrel 30 of adaptor pump 10 in a pre-use state of the adaptor pump 10. Expansion section 32 is typically provided at the proximal end 16 of pressurizing body 12. However, expansion section 32 may be formed at any position along pressurizing body 12 where it would be practical to store plunger 36 in a pre-use state of adaptor pump 10. As shown in FIG. 3, at expansion section 32, the body wall 18 of pressurizing body 12 narrows from a thickness T to a reduced wall thickness Tᵣ. Thus, an inner diameter IDₑₛ of expansion section 32 is larger than an inner diameter ID of the cylindrical barrel 30 of pressurizing body 12. The reduced wall thickness Tᵣ at expansion section 32 allows the body wall 18 of pressurizing body 12 to expand outward in the expansion section 32 under the outward radial force exerted by plunger 36 when seated in the expansion section 32. However, the outward expansion of the body wall 18 in the expansion section 32 is such that an outer diameter ODₑₛ of expansion section 32 of barrel 30 does not become larger than an outer diameter OD of the barrel 30 proximal or forward of expansion section 32.

As further shown in FIG. 3 both an outer surface 38 of the body wall 18 of pressurizing body 12 and an inner surface 40 of the body wall 18 of pressurizing body 12 taper or are stepped to form the reduced wall thickness Tᵣ at expansion section 32. In particular, outer surface 38 of body wall 18 of pressurizing body 12 is tapered or stepped inward toward the central axis L₁ of pressurizing body 12 and inner surface 40 of pressurizing body 12 tapers or is stepped outward away from the central axis L₁ of pressurizing body 12 to form the reduced wall thickness Tᵣ. An alternative configuration to the foregoing is to only taper or step inner surface 40 of pressurizing body 12 outward away from the central axis L of pressurizing body 12. Another alternative is to only taper or step outer surface 38.

The reduced wall thickness Tᵣ at expansion section 32 of barrel 30 accommodates the expansion and plastic creep of the plastic pressurizing body 12 caused by plunger 36 even after long periods of storage. As a result, even after long storage periods, pressurizing body 12 with a pre-positioned plunger 36 may be quickly and easily inserted into front-loading pressure jacket systems, such as that associated with fluid injection apparatus 100 discussed herein. Briefly, during operation of fluid injection apparatus 100, adaptor pump 10 is inserted into a front-loading pressure jacket associated with fluid injection apparatus 100 and plunger 36 is engaged by an injector drive piston of the apparatus 100 which supplies the motive forces to move the plunger 36 from expansion section 32 into barrel 30. Barrel 30 may be referred to as the “working zone” of pressurizing body 12. Proximal lip or flange 34 at the proximal end 16 of pressurizing body 12 typically has an outer diameter that is no greater than the outer diameter OD of barrel 30 of pressurizing body 12 so that the pressurizing body 12 may be smoothly accepted into the front-loading pressure jacket associated with fluid injection apparatus 100 during a loading procedure. As indicated previously, proximal lip 34 also serves to engage corresponding structure associated with fluid injection apparatus 100 to, for example, alert the fluid injection apparatus 100 of the presence of adaptor pump 10 and prevent over-insertion of pressurizing body 12 into the front-loading pressure jacket associated with fluid injection apparatus 100.

Plunger 36 comprises a plunger body 42 having a conical-shaped distal or front end 44 which is shaped to cooperate with conical portion 22 of distal section 20 of pressurizing body 12 and a coupling end 46. The conical end 44 of the plunger body 42 is enclosed, at least in part, by an external elastomeric cover 48 which may be made of rubber, for example. The coupling end 46 of plunger body 42 faces the proximal end 16 of pressurizing body 12 when loaded in barrel 30 of pressurizing body 12. If desired, plunger 36 may be transparent to allow light from a lighted injector drive piston to pass therethrough. In one embodiment, a pair of flexible lugs or coupling members 50 extend outward from coupling end 46 for engaging an injector drive piston associated with fluid injection apparatus 100. The flexible nature of couplings members 50 and their associated flexible engagement with an injector drive piston similar to the injector drive piston associated with apparatus 100 described herein is described in U.S. Pat. Nos. 5,873,861 and 5,947,935, the disclosures of which are incorporated herein by reference. In one form, coupling members 50 are resiliently flexible and may be integrally formed with plunger body 42. In an alternative embodiment, coupling members 50 may be substantially fixed or rigid, as described in U.S. Pat. No. 4,677,980, incorporated herein by reference in its entirety. Coupling members 50 each have an engagement arm 52 for capturing the injector drive piston associated with fluid injection apparatus 100. Coupling members 50 also define a center slot 54 which is configured to receive structure on the injector drive piston, for example, an end plate, which is captured by the engagement arms 52 to connect plunger 36 with the injector drive piston. With plunger 36 disposed within barrel 30, an enclosed interior chamber 56 is defined by the pressurizing body 12 and plunger 36. Luer connector 26 defines a port for loading interior chamber 56 with pressurizing fluid from an external fluid source, such as an inert, motive liquid used as pressurizing hydraulic fluid as described herein.

Adaptor pump 10 further comprises a container body 60 in fluid communication with pressurizing body 12 via a connecting port or passage 62. Connecting port or passage 62 enables passage of pressurizing fluid from pressurizing body 12 to container body 60 as discussed herein. Ideally, container body 60 and pressurizing body 12 are integrally formed as a unitary body connected by a connecting bridge 64 which extends between the container body 60 and pressurizing body 12. Bridge 64 extends longitudinally between container body 64 and pressurizing body 12. Connecting port 62 is defined in bridge 64 and opens to the enclosed interior chamber 56 defined by pressurizing body 12 and plunger 36. Connecting port 62 is desirably provided in the vicinity of alignment flange 28 or just proximal of the alignment flange 28 in barrel 30 of pressurizing body 12. When adaptor pump 10 is in use with fluid injection apparatus 100, connecting port 62 is closed by the plunger body 42 of plunger 36 when the plunger 36 is moved distally into the conical portion 22 of distal section 20 of pressurizing body 12 by the injector drive piston associated with fluid injection apparatus 100 as discussed further herein. Container body 60 has a front or distal end 66 and a rear or proximal end 68. The distal end 66 of container body 60 generally extends or projects over the conical portion 22 of distal section 20 of pressurizing body 12 and over alignment flange 28.

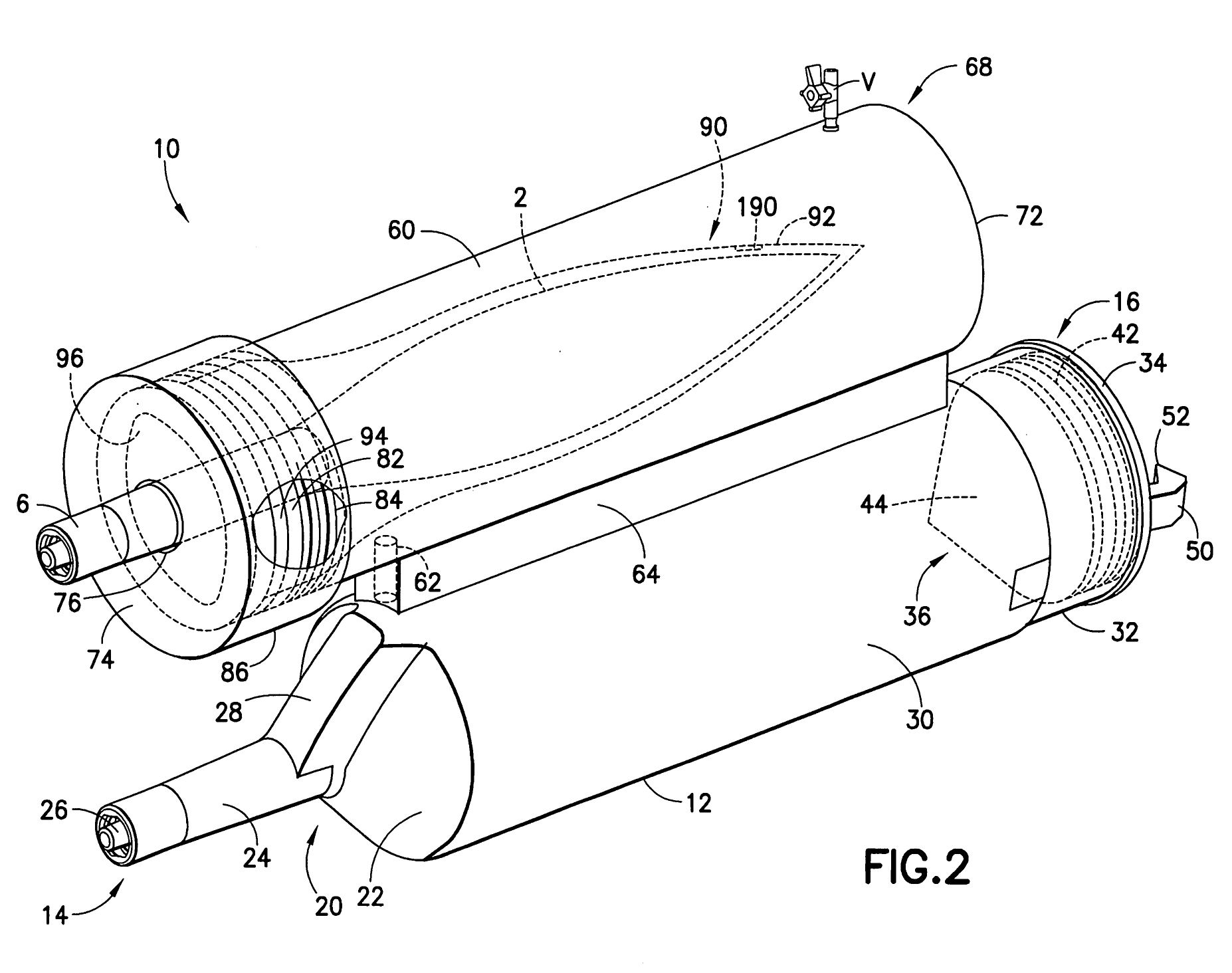

Container body 60 defines an open end or opening 70 at distal end 66 and includes a rear end wall 72 at proximal end 68. Container body 60, in one form, is a cylindrical-shaped container, with open end 70 providing access to the interior of the container body 60. An end closure or cap 74 is used to cover and seal open end 70. Closure 74 defines a central aperture 76 for passage of flexible medical tubing and/or connector(s) such as luer connector(s) associated therewith. Closure 74 and container body 60 are adapted to form a secure mating connection. As an example, container body 60 may define external threads 78 at distal end 66 and closure 74 may define internal threads 80 adapted to engage external threads 78 to form a secure mating connection between closure 74 and container body 60. An annular gasket 82 is interposed between closure 74 and container body 60 to establish a generally fluid-tight seal between closure 74 and container body 60 when closure 74 is threaded onto container body 60. As shown in FIG. 2, annular gasket 82 may be a flat or planar washer-type structure. However, annular gasket 82 may also be in the form of an O-ring type structure which may be disposed in a circumferential groove defined in the outer surface of container body 60 and, for example, around a rim 84 of container body 60 defining open end 70 and which seals against the interior of closure 74 to form a generally fluid-tight seal between closure 74 and container body 60. Other similar fluid-sealing structures may be substituted for the foregoing two examples. As shown in FIG. 3, closure 74 is formed with a depending skirt 86, with internal threads 80 formed or defined on the interior surface of skirt 86.

Container body 60 generally defines an internal receiving chamber 88 which is in fluid communication with the interior chamber 56 defined by pressurizing body 12 and plunger 36 via connecting port 62. Chamber 88 is enclosed by the presence of closure 74 closing the open end 70 defined by the rim 86 of container body 60. In operation of adaptor pump 10, incompressible fluid loaded into interior chamber 56 of pressurizing body 12 is forced into chamber 88 of container body 60 when plunger 36 is moved distally forward in barrel 30 of pressurizing body 12. The forward motion of plunger 36 fills chamber 88 with incompressible fluid and further forward movement of plunger 36 pressurizes the fluid to increase the fluid pressure within chamber 88. As a result, chamber 88 may be referred to as a “pressurizing” chamber 88. Chamber 88 desirably has at least a slightly smaller volume than the interior chamber 56 defined by pressurizing body 12. As a result, a substantially full displacement of plunger 36 from expansion section 32 forward to conical portion 22 of distal section 20 of pressurizing body 12 will fully fill chamber 88 with fluid and pressurizes the chamber 88 to a desired internal hydraulic pressure.

The incompressible fluid and hydraulic pressure supplied to chamber 88 by the action of plunger 36 acts upon a pinch sleeve 90 disposed within chamber 88. Pinch sleeve 90 generally comprises an elastomeric sleeve 92 connected to a generally rigid annular collar 94. Collar 94 is generally sized to engage the rim 84 of container body 60 defining open end or opening 70. Elastomeric sleeve 92 may be secured adhesively or mechanically to collar 94, if desired, and extends into chamber 88. Elastomeric sleeve 92 defines an access opening 96 at a distal or front end and is closed at its opposite end so that elastomeric sleeve 92 forms a receiving pocket 98 which is accessed only through access opening 96. The receiving pocket 98 defined by elastomeric sleeve 92 is generally sized to accept or receive a flexible pouch or bag 2 containing a medical liquid 4 to be injected into a patient. Flexible pouch 2 typically includes a distally-extending coupling structure 6 which is adapted for connection to medical tubing 8. Coupling 6 forms the discharge port of flexible pouch 2. Medical tubing 8 may be connected to an IV needle cannula or a catheter C as illustrated in FIG. 5, discussed herein, which intravenously supplies the medical fluid to the patient. Upon pressurization of chamber 88 by the displacement of plunger 36 in pressurizing body 12, elastomeric sleeve 92 is compressed by the increased hydraulic pressure within chamber 88. This compression in turn acts upon flexible pouch 2 which forces the medical fluid 4 out of flexible pouch 2 via coupling 6. Medical tubing 8 conveys the expelled medical fluid 4 to catheter C for delivery to the patient. As shown in FIG. 3, coupling 6 and/or medical tubing 8 pass through the aperture 76 in closure 74 for connection to catheter C. A fluid-tight seal between coupling 6 and/or medical tubing 8 and aperture 76 in closure 74 is not necessary and may be disadvantageous because clearance around coupling 6 and/or medical tubing in aperture 76 acts as a vent to allow air contained in internal receiving pocket 98 defined by elastomeric sleeve 92 to vent to atmospheric as the elastomeric sleeve 92 is compressed during operation of adaptor pump 10. Suitable medical luer connectors for use in making the fluid connection between coupling 6 and medical tubing 8 and between medical tubing 8 and catheter C may be found in U.S. Patent Application No. 60/741,146, filed Dec. 1, 2005 and entitled “Fluid Deliver System, Fluid Path, and Medical Connector for Use with the Fluid Deliver System and Fluid Path”. The disclosure of U.S. Provisional Patent Application No. 60/741,146 is incorporated herein by reference in its entirety.

Referring to FIGS. 5-9, fluid injection apparatus 100 is shown. In particular, adaptor pump 10 is shown associated with fluid injection apparatus 100. Fluid injection apparatus 100 includes an injector head 102 which may be supported on a support structure (not shown). The injector head 102 has a front end 104 and a faceplate 106 which is attached to or forms the front end 104 of injector head 12. Faceplate 106 generally forms part of the housing of the injector head 102. An injector drive piston 108 is extendable and retractable from injector head 102 through a front opening 110 in faceplate 106. Drive piston 108 includes an end plate 111 adapted to engage the coupling members 50 associated with plunger 36 disposed within pressurizing body 12. The details of injector head 102 and, more particularly, drive piston 108 are described in U.S. Pat. No. 5,383,858 which is hereby incorporated herein by reference in its entirety. Injector drive piston 108 is generally adapted to capture plunger 36 disposed in pressurizing body 12 of adaptor pump 10 by inserting end plate 111 into the center slot 54 defined between coupling members 50 extending from the coupling end 46 of plunger body 42. The flexible coupling members 50 allow engagement arms 52 to deflect around end plate 111 and engage and grasp onto end plate 111. Thereafter, injector drive piston 108 may provide motive forces to plunger 36 to move plunger 36 within the barrel 30 of pressurizing body 12.

A pressure jacket 112 is associated with faceplate 106 and, thus, injector head 102. Pressure jacket 112 associates adaptor pump 10 with the injector head 102 in the present embodiment and positions the adaptor pump 10 to allow drive piston 108 to engage plunger 36 within pressurizing body 12. Generally, pressure jacket 112 extends outward from the front end 104 of injector head 102 and is connected to faceplate 106. Pressure jacket 112 is a generally cylindrical structure defined by a pressure jacket wall 114 and has a front or distal end 116 and a rear or proximal end 118. Pressure jacket 112 defines a receiving bore 120 for receiving pressurizing body 12 of adaptor pump 10 into the pressure jacket 112. The proximal end 118 of pressure jacket 112 engages faceplate 106 to mount the pressure jacket 112 to the faceplate 106 and injector head 102. Generally, the inner diameter of receiving bore 120 is sized to smoothly but snugly receive the outer diameter of pressurizing body 12 of adaptor pump 10. A typical clearance between the outer diameter of pressurizing body 12 and the inner diameter of pressure jacket 112 is about 0.005 inch. Pressure jacket 112 is made of a material capable of restraining the outward expansion of pressurizing body 12 during a pressurization cycle involving adaptor pump 10, as is will known in the medical art. Accordingly, pressure jacket 112 is used to limit the radial expansion of pressurizing body 12. Pressure jacket 112 is desirably formed of clear or slightly opaque molded plastic material to permit viewing and desirably lighting of pressurizing body 12 in the pressure jacket 112.

As indicated previously, pressure jacket 112 is adapted to connect to or engage faceplate 106. In one arrangement, pressure jacket 112 includes a proximal connecting portion 122 which extends in the form of an arc extending along a portion of the circumference of the pressure jacket 112. Connecting portion 122 defines an engagement recess or groove 124 for receiving an engagement tab 126 provided in faceplate 106. The engagement tab 126 is desirably spring-biased in faceplate 106 to radially extend outward from faceplate 106 and engage engagement recess 124, preferably automatically when pressure jacket 112 is connected to or engaged with faceplate 106. The elongated proximal portion 122 at the proximal end 118 of pressure jacket 112 defines an external cam surface 128 which is generally shaped to displace or depress engagement tab 126 radially outward when pressure jacket 112 is connected to faceplate 106. Cam surface 128 defines a generally arcuate distal end or tip that tapers outward in the form of a tapered ramp so that the engagement tab 126 is smoothly displaced or depressed radially outward as pressure jacket 112 is mounted or connected to faceplate 106. The elongated proximal connecting portion 122 of pressure jacket 112 may further define an inner step 130 which is engaged by the rear rim or flange 34 on pressurizing body 12 to prevent over-insertion of the pressurizing body 12 in pressure jacket 112.

Pressure jacket 112 is formed with a light diffusing device or structure 132 which is formed as part of the pressure jacket wall 114 or outer surface of the pressure jacket 112. Light diffusing structure 132 is generally formed as a beveled surface on the pressure jacket wall 114 of pressure jacket 112 and is adapted to diffuse light entering pressure jacket 112 from one or more light sources associated with faceplate 106 as discussed herein. The beveled surface forming light diffusing structure 132 is located substantially opposite from the light sources associated with faceplate 106 when the pressure jacket 112 is connected to the faceplate 106 as discussed further herein. The beveled surface on pressure jacket wall 114 forms a diffusing lens or structure on the body of the pressure jacket 112 which directs and diffuses light into pressurizing body 12 from the light sources disposed on faceplate 106 for providing effective viewing of the contents of pressurizing body 12. The beveled surface does not need to extend completely about the circumference of the pressure jacket wall 114 and need only extend partially about the circumference of the pressure jacket wall 114, generally in the vicinity of the light sources associated with faceplate 106. Thus, the pressure jacket wall 114 of pressure jacket 112 could be formed with multiple beveled surfaces.

Pressure jacket 112 defines a longitudinal slot 134 located to receive the bridge 64 connecting pressurizing body 12 and container body 60. Thus, bridge 64 serves as a key structure for properly aligning the adaptor pump 10 to permit insertion of pressurizing body 12 into the receiving bore 120 in pressure jacket 112. Additionally, since connecting bridge 64 is generally aligned with alignment flange 28 on pressurizing body 12, bridge 64 likewise orients the pressurizing body 12 in a proper orientation to allow injector drive piston 108 to engage the coupling members 50 extending proximally from plunger 36 disposed within the pressurizing body 12. Only one loading orientation is permitted by the cooperative engagement between bridge 64 and bridge receiving slot 134.

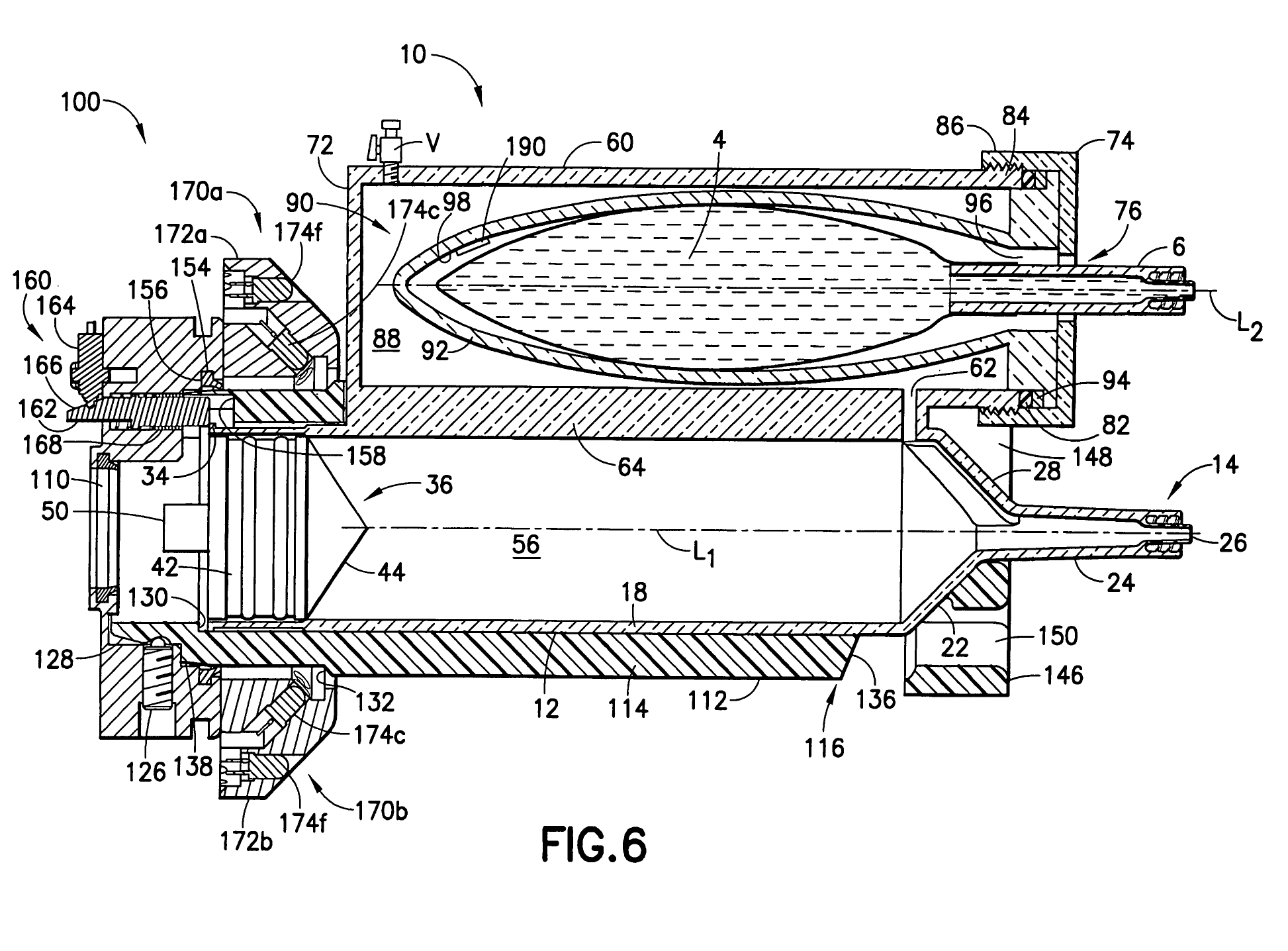

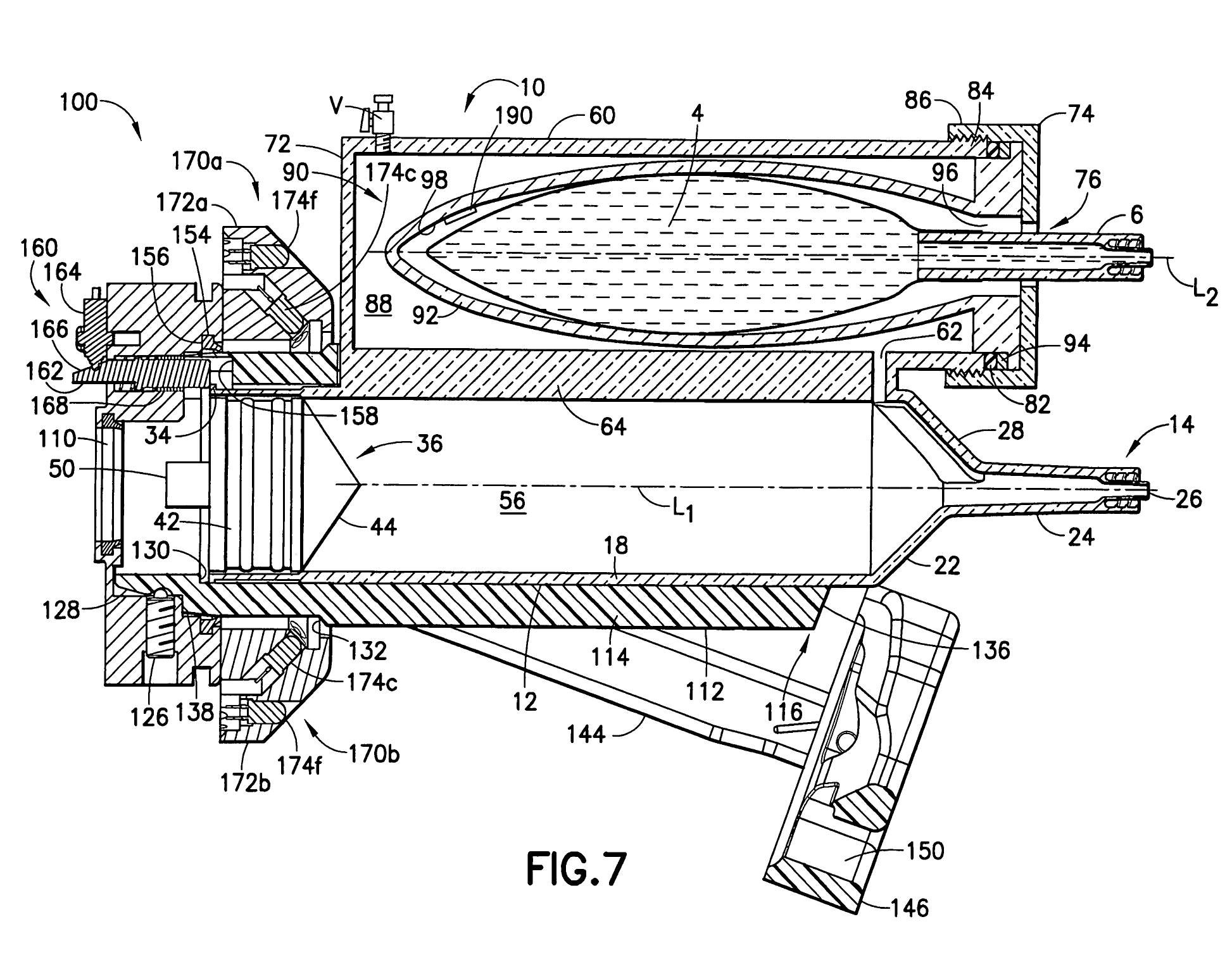

The distal end 116 of pressure jacket 112 defines a beveled portion or surface 136. Beveled portion 136 generally comprises approximately half of the circumference of the distal end 116 of the pressure jacket 112. The function of beveled portion 136 or surface is discussed herein. Further, as shown in FIGS. 6-7, the proximal connecting portion 122 of pressure jacket 112 is formed with a tapered proximal outer surface 138 which extends circumferentially about the proximal connecting portion 122 and which aids in guiding the proximal end 118 of pressure jacket 112 into engagement with faceplate 106.

Fluid injection apparatus 100 further comprises a support structure 140 which extends outward from faceplate 106 on injector head 102 and provides support for adaptor pump 10 when associated with the fluid injection apparatus 100. Mechanical details and operational characteristics of support structure 140 are discussed in U.S. patent application Ser. No. 10/818,477, filed Apr. 4, 2005 and entitled “Fluid Injection Apparatus With Front Load Pressure Jacket, Light Illumination, And Syringe Sensor”. The disclosure of U.S. patent application Ser. No. 10/818,477 is hereby incorporated by reference in its entirety. Generally, support structure 140 comprises at least one, and desirably two, support arms 142, 144 extending outward from faceplate 106. Support arms 142, 144 extend through respective front openings (not shown) defined in faceplate 106. Support arms 142, 144 are interconnected at their distal ends by a retaining member 146. Retaining member 146 is pivotally connected to respective support arms 142, 144 to allow pivotal movement relative to the support arms 142, 144. Retaining member 146 defines a central receiving slot 148 which is substantially vertically oriented and is configured to receive and support the elongated neck 24 extending from the conical portion 22 of distal section 20 of pressurizing body 12. Retaining member 146 defines one or more openings 150 which are spaced radially outward from receiving slot 148. Receiving slot 148 and openings 150 permit an operator of the fluid injection apparatus 100 to view the distal section 20 of pressurizing body 12 during use of adaptor pump 10 in apparatus 100.

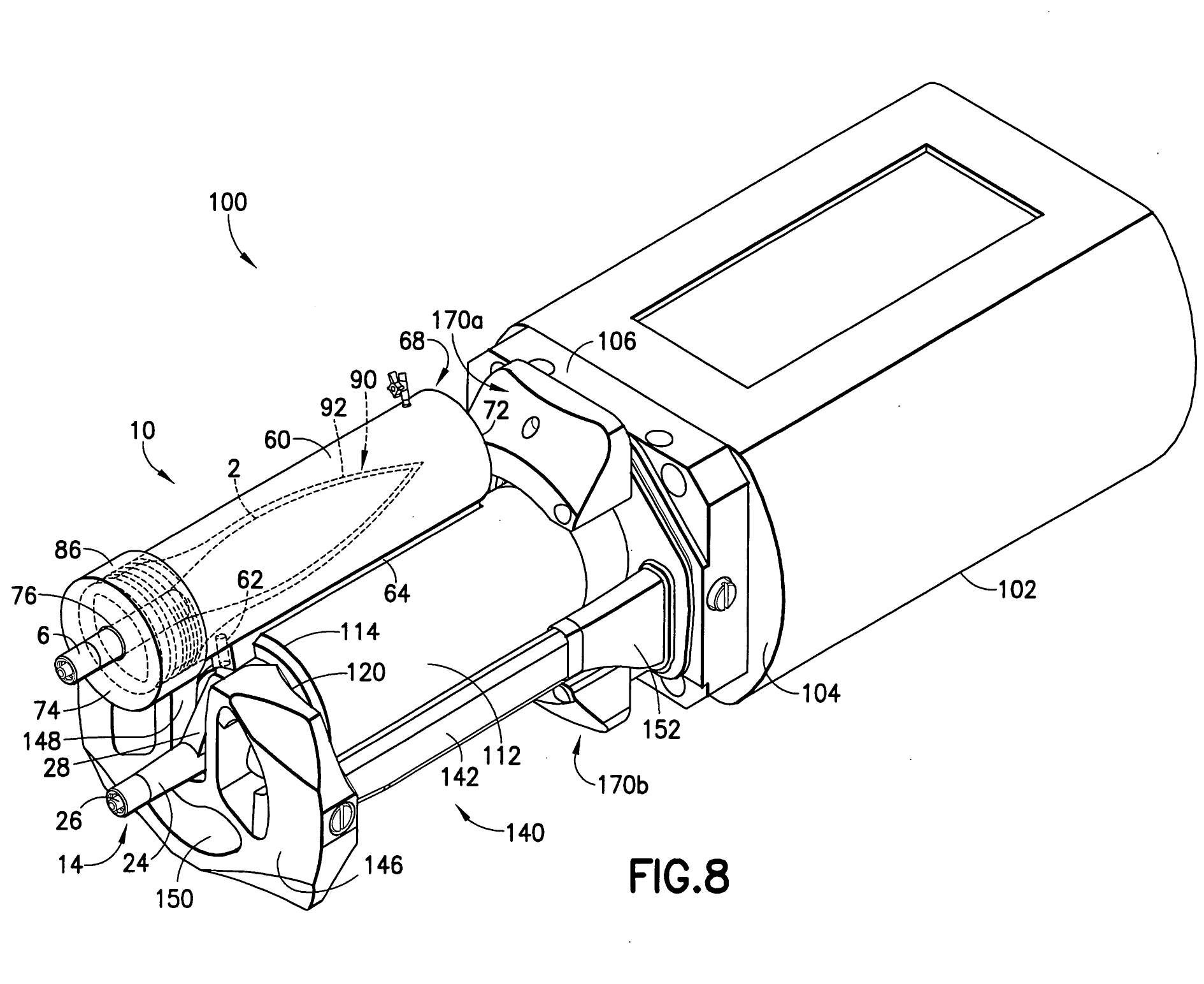

Support arms 142, 144 are generally configured to be movable between a first position (FIG. 8) wherein retaining member 106 receives the elongated neck 24 extending from the conical portion 22 of distal section 20 of pressurizing body 12 and cooperates with the conical portion 22 and prevents removal of adaptor pump 10 from pressure jacket 112, and a second, pivoted position (FIG. 7) wherein the elongated neck 24 is disengaged sufficiently from receiving slot 148 and the conical portion 22 is sufficiently disengaged from the retaining member 106 to allow removal of pressurizing body 12 from the pressure jacket 112. In the second, pivoted position of support arms 142, 144; the support arms 142, 144; and retaining member 146 are spaced a distance below pressure jacket 112 and adaptor pump 10. With support arms 142, 144 in the first position, support structure 140 is in an engaged or restraining position. When support arms 142, 144 are moved to the second position, support structure 140 is in a disengaged or removal-permitting position or configuration. An elastomeric sleeve 152 may be provided around each support arm 142, 144 which covers and seals the openings (not shown) in faceplate 106 and through which the support arms 142, 144 extend.

Faceplate 106 associated with injector hear 102 is adapted to secure the connection of pressure jacket 112 thereto. Pressure jacket 112 is secured to faceplate 106 by the engagement of engagement tab 126 disposed within faceplate 106 with corresponding engagement recess 124 formed generally at the proximal end 118 of pressure jacket 112. As described previously, the proximal end 118 of pressure jacket 112 is formed with a generally elongated proximal connecting portion 122. Engagement recess 124 is formed in proximal connecting portion 122 and is in the form of a groove in the elongated proximal connecting portion 122. Engagement tab 126 is spring-biased in faceplate 106 to radially extend outward and engage the engagement recess 124 automatically when pressure jacket 112 is connected to faceplate 106. External cam surface 128 on proximal connecting portion 122 is generally shaped to displace and depress engagement tab 126 radially outward when pressure jacket 112 is mounted or connected to faceplate 106. Cam surface 128 defines a generally arcuate distal end or tip that tapers outward in the form of a tapered ramp so that engagement tab 126 is smoothly displaced or depressed radially outward as pressure jacket 112 is mounted or connected to faceplate 106.

As described previously, tapered proximal outer surface 138 extends circumferentially about the proximal end 118 of pressure jacket 112 and aids in guiding the proximal end 118 of pressure jacket 112 into engagement with faceplate 106. Additionally, a sealing bushing 154 is provided in a circumferential recess or groove 156 formed in faceplate 106. Sealing bushing 154 is generally adapted to engage the tapered proximal outer surface 138. Sealing bushing 154 forms a barrier which substantially prevents contaminants and liquids from entering front opening 110 in faceplate 106.

The proximal end 118 of pressure jacket 112 further defines an internal recess portion 158 for accommodating a sensor 160. Internal recess portion 158 is generally configured to accommodate sensor 160 without causing activation of the sensor 160 when pressure jacket 112 is connected to faceplate 106. Sensor 160 is a two-piece component comprised of an actuating member or pin 162 and an electrical switch 164. A suitable model for electric switch 164 is manufactured by Omron. Actuating member 162 includes a tapered proximal surface 166 in engagement or contact with electrical switch 164 and is adapted to activate the electrical switch 164 once pressurizing body 12 is loaded into pressure jacket 112 and engages actuating member 162. In particular, when pressurizing body 12 is loaded into pressure jacket 112, the proximal end 16 of the pressurizing body 12 engages or contacts actuating member 162. In operation, as pressurizing body 12 is initially inserted into pressure jacket 112 during a loading operation, flange 34 on the proximal end 16 of the pressurizing body 12 will initially contact actuating member 162. When pressurizing body 12 is fully inserted into pressure jacket 112 and secured in place in the pressure jacket 112 by the components of support structure 140, the proximal end 16 of the pressurizing body 12 will move actuating member 162 proximally into faceplate 106 which causes the tapered proximal surface 166 of the actuating member 162 to move proximally. The increased thickness of actuating member 162 provided by the tapered proximal surface 166 depresses and activates electrical switch 164. Electrical switch 164 is adapted to generate a signal indicating the presence of pressurizing body 12 in pressure jacket 112 which could be used as a control signal by injector head 102 indicating, for example, that pressurizing body 12 may now be filled with fluid. Actuating member 162 is normally biased, for example, by a spring 168, to a non-activating position relative to electrical switch 164. The biasing force acting on actuating member 162 is overcome when pressurizing body 12 is loaded into pressure jacket 112 and secured in place therein by the elements discussed previously.

As will be clear from viewing FIGS. 6-7, proximal lip 34 on pressurizing body 12 is adapted to engage or contact actuating member 162 when the pressurizing body 12 is loaded into pressure jacket 112. The raised proximal lip 34 provides a sufficient outward-extending proximal structure at the proximal end 14 of pressurizing body 12 to enable the pressurizing body 12 to engage actuating member 162 and depress or move the actuating member 162 proximally to activate electrical switch 164. However, as discussed previously, proximal lip 34 preferably has an outer diameter that is no greater than the outer diameter of barrel 30. Moreover, as illustrated in FIGS. 6-7, proximal lip 34 may be used as a stop structure to engage inner step 130 defined within pressure jacket 112 which will serve as a limiter to prevent over-insertion of pressurizing body 12 into the pressure jacket 112.

Another feature of injector head 102 relates to a lighting arrangement for illuminating pressure jacket 112 and adaptor pump 10 when the pressurizing body 12 is loaded into the pressure jacket 112. The lighting arrangement for injector head 102 comprises at least one and typically a pair of opposing light sources 170a, 170b, which are located on opposite top and bottom sides of pressure jacket 112 of the pressure jacket 112. In particular, opposing light sources 170a, 170b comprise respective light source housings 172a, 172b each having internally positioned lights 174, such as light-emitting diodes (LED's) or similar light-emitting elements. Light source housings 172a, 172b may be connected to faceplate 106 by any suitable mechanical or adhesive means. While light source housings 172a, 172b are illustrated as being located on the top and bottom of faceplate 106 in the views shown in FIGS. 5-9, other configurations, such as locating the light source housings 172a, 172b on opposing lateral sides of faceplate 106, are also possible. Additionally, only one of light sources 170a, 170b, such as upper light source 170a, is required to illuminate pressure jacket 112 and adaptor pump 10 associated therewith.

The respective light source housings 172a, 172b each include a plurality of lights 174. In the illustrated arrangement, the respective light source housings 172a, 172b include at least one inward-directed or angled center light 174_{c}. Angled center lights 174_{c} illuminate pressure jacket 112 on opposing top and bottom sides thereof, with the light transmitted through the pressure jacket 112 being diffused by the pressure jacket 112 into pressurizing body 12 loaded into the pressure jacket 112. The respective housings 172a, 172b may further include a plurality of forward directed lights 174_{f}. The forward directed lights 174_{f} in upper light source housing 172a are used to illuminate container body 60 generally along a central longitudinal axis L₂ thereof. The forward directed lights 174_{f}in lower light source housing 170b may be used for other purposes such as indicating when the injector head 102 is ready to begin an injection procedure using adaptor pump 10.

As described previously, pressure jacket 112 is desirably formed with a light diffusing device or structure 132 which is formed or provided on the pressure jacket wall 114. In one form, the light diffusing structure 132 is a beveled surface on the pressure jacket wall 114 of pressure jacket 112. Light diffusing structure 132 is formed or provided on the pressure jacket wall 114 of pressure jacket 112 so as to be located substantially opposite from the angled light sources 174, disposed within the respective light source housings 172a, 172b of light sources 170a, 170b when pressure jacket 112 is mounted to faceplate 106. The beveled surface forming light diffusing structure 132 does not need to extend completely about the circumference of pressure jacket 112, and need only extend partially about the circumference of the pressure jacket 112 typically in the vicinity of the top and bottom light sources 170a, 170b. Thus, pressure jacket 112 could be formed with two beveled surfaces located on opposite sides of pressure jacket 112 and which would be substantially coincident with the light emitted by the angled light sources 174, disposed within the respective light source housings 172a, 172b of light sources 170a, 170b top when the pressure jacket 112 is mounted to faceplate 112. Light diffusing structure 132 on the pressure jacket wall 114 of pressure jacket 112 forms a light-diffusing lens or structure on the body of the pressure jacket 112 which diffuses or, more clearly, directs and diffuses light into pressurizing body 12 from light sources 170a, 170b for providing effective viewing of the contents of pressurizing body 12.

Referring to FIGS. 1-9, operation of fluid injection apparatus 100 and adaptor pump 10 associated therewith will now be discussed. It is assumed for the sake of a convenient starting point to discuss the operational association of adaptor pump 10 with fluid injection apparatus 100 that pressure jacket 112 is already mounted or connected to faceplate 106. Briefly, pressure jacket 112 is mounted to faceplate 106 by inserting the proximal end 118 of pressure jacket 112 into the front opening 110 in the faceplate 106. As proximal end 118 of pressure jacket 112 is inserted into faceplate 106, external cam surface 128 on proximal connecting portion 122 at the proximal end 118 of the pressure jacket 112 displaces and depresses engagement tab 126 radially outward. Engagement tab 126 is spring-biased and automatically engages engagement groove 124 in proximal connecting portion 122 as the proximal end 118 of pressure jacket 112 is inserted fully into faceplate 106. Once pressure jacket 112 is mounted to faceplate 106 by the engagement of engagement tab 126 in engagement groove 124, adaptor pump 10 may be inserted into the pressure jacket 112 to associate the adaptor pump 10 with fluid injection apparatus 100.

Adaptor pump 10 is associated with fluid injection apparatus 100 by inserting pressurizing body 12 into receiving bore 120 defined by pressure jacket 112. To insert pressurizing body 12 into pressure jacket 112, bridge 64 connecting pressurizing body 12 and container body 60 must be aligned with bridge slot 134 defined in pressure jacket wall 114. It will be apparent that support structure 140 must be in a disengaged or pivoted position wherein support arms 142, 144 and retaining member 146 are pivoted to a position below pressure jacket 112 to allow the insertion of pressurizing body 12 into the receiving bore 120 in the pressure jacket 112. Bridge 64 is aligned with alignment flange 28, and bridge 64 and alignment flange 28 desirably provide a visible indication of the orientation of slot 54 between coupling members 50 extending proximally from the coupling end 46 of plunger body 42 of plunger 36 seated in pressurizing body 12. As indicated previously, only one loading orientation is permitted by the cooperative “keyed” engagement between bridge 64 and bridge slot 134. Accordingly, it is desirable to have plunger 36 orientated in pressurizing body 12 such that the slot 54 defined between coupling members 50 is aligned to automatically accept end plate 111 on injector drive piston 108 when the injector drive piston 108 is extended from injector head 102 and through faceplate 106.

As pressurizing body 12 is inserted into pressure jacket 112, proximal flange or lip 34 at the proximal end 16 of pressurizing body 12 contacts and depresses sensor 160 which operates in the manner described previously to alert, for example, a control unit in injector head 102 of the presence of pressurizing body 12 in pressure jacket 112 and, thus, the association of adaptor pump 10 with fluid injection apparatus 100. Once pressure jacket 112 is mounted to faceplate 106 and pressurizing body 12 is fully inserted into the pressure jacket 112, light sources 170a, 170b on faceplate 106 are oriented to direct light into pressurizing body 12 through pressure jacket 112. The light diffusing structure 132 (i.e., beveled surface) on pressure jacket 112 is automatically aligned with angled light emitting elements 174_{c} when mounted to faceplate 106. Light diffusing structure 132 operates as described previously to diffuse light from angled light emitting elements 174_{c} into pressurizing body 12. Likewise, the forward-directed light emitting elements 174_{f} associated with the “top” light source 170a on faceplate 106 are directed to light container body 60 through rear end wall 72 of container body 60. Supporting structure 140 may be returned to an engaged position wherein retaining member 146 cooperates with the conical portion 22 of distal section 20 of pressurizing body 12 and elongated neck 24 of the distal section 20 is disposed in receiving slot 148.

Turning now to an exemplary set-up procedure for container body 60, container body 60 is initially provided with closure 74 removed from the distal end 66 and rim 84 of container body 60. Thus, chamber 88 defined by container body 60 is accessible through open end or opening 70 in container body 60. Container body 60 is prepared for a fluid injection procedure involving adaptor pump 10 by placing sealing gasket 82 on rim 84 of container body 60 and then inserting pinch sleeve 90 into the container body 60 through open end or opening 70. In particular, pinch sleeve 90 is associated with container body 60 by inserting the elastomeric sleeve 92 of pinch sleeve 90 into the chamber 88 through opening 70. Rigid annular collar 94 connected to elastomeric sleeve 92 is placed in engagement or contact with sealing gasket 82. Sealing gasket 82 is formed of elastomeric material which is compressed between collar 94 and rim 84 when closure 74 is place over rim 84 and secured to the distal end 66 of container body 60. Once elastomeric sleeve 92 is placed within chamber 88, internal pocket 98 defined by elastomeric sleeve 92 is ready to accept a fluid-filled flexible pouch 2 via access opening 96 in elastomeric sleeve 92. Elastomeric sleeve 92 is desirably sized to smoothly by snugly receive fluid-filled flexible pouch 2 which is filled with medical fluid 4 to be injected into a patient. Coupling 6 and/or flexible medical tubing 8 extending from pouch 2 is inserted through aperture 76 in closure 74 to allow the closure 74 to be secured (i.e., threaded) onto the distal end 66 of container body 60. As illustrated in several of FIGS. 1-9, the distal end 66 of container body 60 extends or cantilevers over the conical portion 22 and the elongated neck 24 of distal section 20 of pressurizing body 12. Bridge 64 connecting pressurizing body 12 and container body 60 raises the distal end 66 of container body 60 above these formations on pressurizing body 12 so that closure 74 may be easily threaded onto the distal end 66 of container body 60. As described previously, a threaded connection between closure 74 and container body 60 is just one example of a secure connection between these structures and may be replaced by suitably equivalent mechanical connections. A threaded connection has the advantage of a simplified method of applying compressive pressure on annular gasket 82 so that the annular gasket 82 provides a generally fluid-tight seal between the rim 84 of container body 60 and the collar 94 of pinch sleeve 90, which results in a generally fluid-tight and enclosed chamber 88.

A pouch sensor 190 may be associated with pinch sleeve 90 and, in particular, elastomeric sleeve 92. Pouch sensor 190 is adapted to identify at least the presence of a pouch 2 in elastomeric sleeve 92 once inserted therein by a user of fluid injection apparatus 100. However, pouch sensor 190 may be able to identify other characteristics of pouch 2 like the contents of pouch 2, expiration date of the contents, etc., as discussed further herein. In particular, pouch sensor 190 is disposed within receiving pocket 98 defined by elastomeric sleeve 92. Pouch sensor 190 is generally adapted to read a corresponding encoding device 192 disposed on the body of pouch 2 and, ideally, is further adapted to transmit information “read” from pouch 2 to a control unit. As examples, such a control unit may be disposed internally within injector head 102 or be a remotely positioned control unit used to control operation of injector head 102. Pouch sensor 190 may be hardwired to the control unit or may be able to communicate with the control unit wirelessly to convey data or signals to the control unit. As a result, such a control unit is able to ascertain from the received signal or signals at least the presence of a pouch 2 in pinch sleeve 90. However, it is desirable to provide additional encoded information in the encoding device 192 so that the signal(s) from pouch sensor 190 contains additional information which may used, for example, as programming inputs to the control unit. For example, pouch sensor 190 may able to convey information which could be used as programming inputs to the control unit which will thereafter direct fluid injection apparatus 100 to perform a fluid delivery/injection procedure in accordance with the detected information. These inputs may be unique and tailored to the specific fluid injection procedure to be performed on a specific patient.

In the present instance, encoding device 192 on pouch 2 is a simple bar code which is optically read by pouch sensor 190. As an alternative to encoding device 192 being a bar code, the encoding device 192 may be a series of indented or raised spaced bars provided on the body of pouch 2 or a mechanically read device, such as a projection on pouch 2 which registers with a switch or other electromechanical structure provided in receiving pocket 98. Another alternative is to provide encoding device 192 as a bar code, the encoding device may consist of characters, dots, geometric shapes, etc. that are read optically in generally the same manner as a bar code by pouch sensor 190.

Pouch sensor 190 is positioned in receiving pocket 98 of elastomeric sleeve 92 to “automatically” read encoding device 192 on pouch 2 once the pouch is inserted into the elastomeric sleeve. When pouch sensor 190 “reads” encoding device 192 it then sends a signal or signals to the control unit, discussed previously, which then recognizes, typically at a minimum, the presence of pouch 2. Desirably, other information is contained in this signal or signals. In one example, the signal could include “operation” information which is interpreted by the control unit and used to program or modify the programming of injector head 102 and, thus, the operation of fluid injection apparatus 100 generally. However, more basic or routine information relating to pouch 2 may also be included in the signal or signals. Examples of such pouch-related information includes the dimensions (i.e., volume) of pouch 2, contents of pouch 2, expiration date of the contents, warning information relating to the contents, general safety information, etc.

With pouch 2 disposed within receiving pocket 98 defined by elastomeric sleeve 92 of pinch sleeve 90, the pouch 2 may be connected to catheter C or another patient interface device, such as an IV needle cannula, for delivering the medical fluid 4 contained in pouch 2 to a patient. Suitable luer connectors and flexible medical tubing sections including appropriate luer connectors for making the fluid connections between coupling 6 and/or flexible medical tubing 8 to catheter C were identified previously. Adaptor pump 10 may now be loaded with an inert incompressible fluid, such as water, (hereinafter “pressurizing fluid”) which is used to pressurize chamber 88 and apply compressive hydraulic pressure to pinch sleeve 90 and fluid-filled pouch 2 disposed therein. The pressurizing fluid is contained in an external fluid supply source 180 such as a fluid-filled IV pouch 182. External fluid supply source 180 is connected by flexible medical tubing 184 to luer connector 26 on the elongated neck 24 of distal section 20 of pressurizing body 12. It is desirable that a fluid source connector 186 be provided at the end of flexible medical tubing 184, for example, in the form of a two-way stopcock which may be operated to alternately place the interior chamber 56 defined by pressurizing body 12 and plunger 36 in fluid communication with the fluid supply source 180 and close and seal luer connector 26 on the elongated neck 24 of distal section 20 of pressurizing body 12. For example, once pressurizing body 12 and, in particular, the interior chamber 56 defined by pressurizing body 12 and plunger 36 is filled with pressurizing fluid from fluid supply source 180, fluid source connector (i.e., two-way stopcock) 186 may be closed to seal the interior chamber 56.

Pressurizing fluid from fluid supply source 180 is loaded into adaptor pump 10 according to the following exemplary procedure which may have various alternative steps and modifications. Prior to connecting luer connector 26 to fluid source connector 186, it is desirable to operate injector head 102 to extend injector drive piston 108 to engage coupling members 150 on plunger body 42 of plunger 36 and move the plunger 36 to a distal position wherein conical end 44 of the plunger body 42 is seated in conical portion 22 of distal section 20 of pressurizing body 12. Fluid source connector 186 may then be connected to luer connector 26 at the distal end of elongated neck 24 of distal section 20 of pressurizing body 12. Plunger 36 is then be drawn proximally in barrel 30 of pressurizing body 12 which draws in pressurizing fluid from fluid supply source 180. Fluid supply source 180 may be elevated above adaptor pump 10 to allow a pressure head differential to assist in loading pressurizing fluid from fluid supply source 180 into pressurizing body 12 and interior chamber 56 therein. As plunger 36 is retracted proximally in barrel 30, pressurizing fluid from fluid supply source 180 desirably fully fills interior chamber 56 within pressurizing body 12. Once interior chamber 56 of pressurizing body 12 is substantially to fully filled with pressurizing fluid from fluid supply source 180, fluid source connector 186, typically a two-way stopcock, may be operated to a closed position closing and sealing luer connector 26 and isolating interior the interior chamber 56 from the fluid supply source 180. The pressurizing fluid now loaded into interior chamber 56 is now ready to be supplied to container body 60 to fill and pressurize chamber 88 in container body 60.

Pressurizing fluid is provided to container body 60 by displacing plunger 36 distally or forward in barrel 30 of pressurizing body 12 using injector drive piston 108. As plunger 36 moves forward, pressurizing fluid enters chamber 88 within container body 60 via connecting port 62 in bridge 64. Continued forward or distal movement of plunger 36 substantially to fully fills chamber 88 with pressurizing fluid. A vent V is desirably provided at the top end of container body 60, and which is opened by the operator of fluid injection apparatus 100 to allow residual air trapped within container body 60 to escape as chamber 88 is filled with pressurizing fluid. Once pressurizing fluid substantially fills chamber 88, additional pressurizing fluid pumped into the chamber 88 by the forward movement of plunger 36 in barrel 30 of pressurizing body 12 the hydraulic pressure in chamber 88 increases proportionally and acts upon the outer surface of elastomeric sleeve 92 of pinch sleeve 90. At this point, vent V is typically operated to a closed position to seal chamber 88. As an example, vent V may be a two-way stopcock that is operated to a closed position to seal chamber 88 and opened to allow, for example, the escape of trapped air in the chamber 88. The compressive hydraulic pressure acting one elastomeric sleeve 92 is applied to pouch 2 contained within receiving pocket 96 defined by elastomeric sleeve 92. It will be apparent that the interior of receiving pocket 96 is fluid-isolated from chamber 88. The compressive force applied by elastomeric sleeve 92 to pouch 2 forces medical fluid 4 in pouch 2 out through coupling 6 and medical tubing 8. The medical fluid 4 is expelled from pouch 2 at a regulated rate, approximately equivalent to the rate at which pressurizing fluid is pumped into pressurization chamber 88.

Once the contents of pouch 2 are fully expelled, the forward movement of injector drive piston 108 moving plunger 36 is halted and vent V at the top end of container body 60 is opened. Injector drive piston 108 may then be operated in reverse to retract plunger 36 in barrel 30 of pressurizing body 12 causing the pressurizing fluid in chamber 88 to be drawn back into the interior chamber 56 within the pressurizing body 12. Once container body 60 is substantially emptied of fluid, fluid source connector 186, typically a two-way stopcock, may be opened to reestablish fluid communication between interior chamber 56 and fluid supply source 180. If desired, fluid supply source 180 may be re-positioned to an elevation below pressurizing body 12 so that pressurizing fluid drawn back into interior chamber 56 may emptied into fluid supply source 180. As an alternative, fluid source connector 186 may disconnected entirely from luer connector 26 at the distal end of elongated neck 24 of pressurizing body 12, allowing the pressurizing fluid in interior chamber 56 to drain into a suitable waste receptacle. The injector head 102 may be tipped on pivot to assist in this latter process.

With pressurizing fluid now emptied from adaptor pump 10, closure 74 on container body 60 is removed. It is generally desirable to disconnect catheter C from medical tubing 8 and/or coupling 6 or possibly disconnect medical tubing 8 from coupling 6 before removing closure 74 from container body 60. Closure 74 is then unthreaded from engagement with the distal end 66 of container body 60 and removed. The empty flexible pouch 2 is then removed from the receiving pocket 96 in elastomeric sleeve 92 of pinch sleeve 90. Pinch sleeve 90 and annular gasket 82 may then be removed from association with container body 60. Pinch sleeve 90 and annular gasket 82 may be discarded or reused as desired. Typically, these components do not require re-sterilization since the medical fluid 4 in flexible pouch 2 remains fluid-isolated from pinch sleeve 90 throughout the operation of adaptor pump 10. Accordingly, these components may be reused in subsequent fluid injection procedures involving fluid injection apparatus 100 and adaptor pump 10 which itself may be reused in subsequent procedures. However, if spillage of blood or other bodily fluids contacts adaptor pump 10 or any component thereof, such as pinch sleeve 90, closure 74, etc., adaptor pump 10 and/or any such individual components may be discarded as desired and a new adaptor pump 10 loaded into fluid injection apparatus 100.

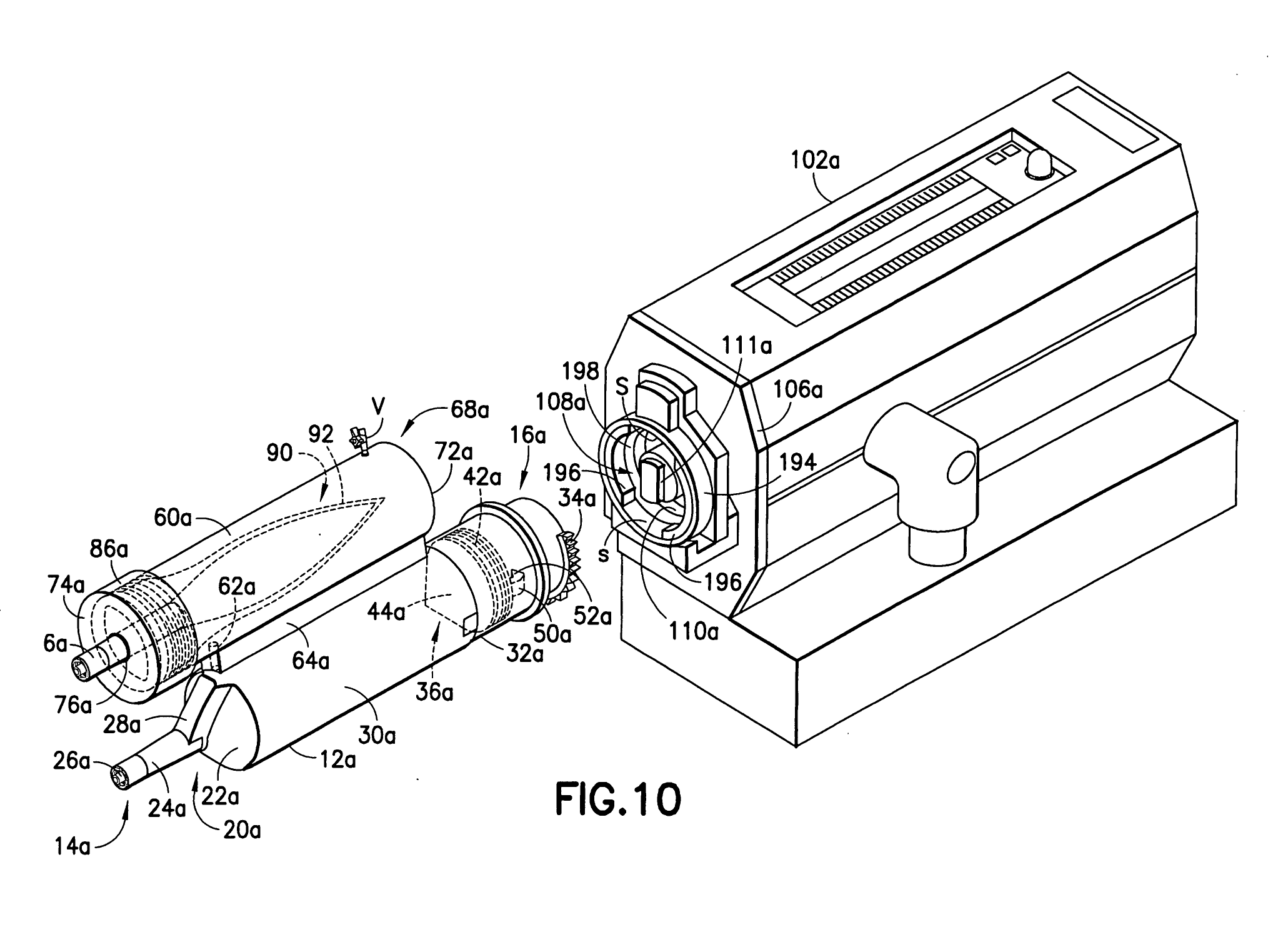

Referring to FIG. 10, another embodiment of fluid injection apparatus 100a is shown. In the disclosed embodiment, adaptor pump 10 is directly associated or engaged with injector head 102a and, in particular, faceplate 106a of injector head 102a. Accordingly, pressure jacket 112 and support structure 140 discussed previously are not required to associate adaptor pump 10a with injector head 102a. The particulars of injector head 102a is described in U.S. Pat. No. 5,383,858 to Reilly et al. which was previously incorporated by reference. As with injector head 102 discussed previously, injector head 102a comprises an extendable drive piston 108a with an end plate 111a. Drive piston 108a is extendable through front opening 110a in faceplate 106a to engage pressurizing plunger 36a seated in pressurizing body 12a of adaptor pump 10a.

Adaptor pump 10a is identical in all respects to adaptor pump 10 discussed previously but includes certain modifications to pressurizing body 12a to allow pressurizing body 12a to associate or engage directly with injector head 102a. In particular, the proximal end 16a of pressurizing body 12a is formed with a pair of flanges 34a disposed on opposite lateral sides of pressurizing body 12a and are adapted for engagement with faceplate 106a forming the front end 104a of injector head 102a. Faceplate 106a is formed with a cylindrical extension 194 which includes opposing retaining flanges 196 which define a pair of arcuate slots S therebetween for accepting or receiving the opposed flanges 34a on pressurizing body 12a. In use, flanges 34a are aligned with slots S to allow the flanges 34a to be inserted into the cylindrical extension 194 past retaining flanges 196, and into a groove or annular ring 198 defined behind retaining flanges 196. Pressurizing body 12a is then rotated, for example clockwise, so that opposed flanges 34a on pressurizing body 12a become engaged behind retaining flanges 196 and are seated in groove 198 to secure the pressurizing body 12a to faceplate 106a of injector head 102a. The rotation of pressurizing body 12a is preferably limited by suitable stops (not shown) in groove or annular ring 198. Other than the foregoing differences associated with the proximal end 16a of pressurizing body 12a, pump adaptor 10a is identical in all other respects to pump adaptor 10 discussed previously. However, as indicated, pump adaptor 10a now cooperates or engages directly with injector head 102a rather than being supported thereto by a pressure jacket and associated support structure as described previously in connection with fluid injection apparatus 100.

While the present invention was described by way of a detailed description of several embodiments of a fluid injection apparatus and adaptor pump therefor, those skilled in the art may make modifications and alterations to this invention without departing from the scope and spirit of the invention. Accordingly, the foregoing description is intended to be illustrative rather than restrictive. The invention described hereinabove is defined by the appended claims, and all changes to the invention that fall within the meaning and the range of equivalency of the claims are to be embraced within their scope.

1. An adaptor pump comprising:
a pressurizing body;
a plunger seated in the pressurizing body;
a container body defining an internal chamber in fluid communication with the interior of the pressurizing body, the container body defining and opening allowing access to the internal chamber;
a pinch sleeve inserted in the container body opening and disposed in the internal chamber, the pinch sleeve adapted to receive a flexible pouch containing medical liquid; and
a closure closing the container body opening and enclosing the pinch sleeve in the internal chamber.

2. An adaptor pump as claimed in claim 1 wherein the pressurizing body and container body are integrally formed of molded plastic material.

3. An adaptor pump as claimed in claim 1 wherein the pressurizing body and container body are connected by a bridge.

4. An adaptor pump as claimed in claim 3 wherein the interior of the pressurizing body and internal chamber are in fluid communication via a port defined in the bridge.

5. An adaptor pump as claimed in claim 1 wherein the pinch sleeve comprises an elastomeric sleeve shaped to receive the flexible pouch.

6. An adaptor pump as claimed in claim 1 wherein the closure defines an aperture for passage of medical tubing for connection to a discharge port on the flexible pouch.

7-8. (canceled)

9. An adaptor pump as claimed in claim 1 further comprising a pouch sensor disposed in the pinch sleeve.

10. A fluid injection apparatus comprising:
a pressurizing device adapted for fluid connection to an incompressible fluid source;
a container body defining an internal chamber in fluid communication with the pressurizing device, the container body defining an opening allowing access to the internal chamber;
a pinch sleeve inserted in the container body opening and disposed in the internal chamber, the pinch sleeve adapted to receive a flexible pouch containing medical liquid and comprising a discharge port; and
a closure closing the container body opening and enclosing the pinch sleeve in the internal chamber;
wherein the pressurizing device is adapted to pump incompressible fluid into the internal chamber such that increasing hydraulic pressure in the internal chamber squeezes the pinch sleeve to expel medical fluid from the flexible pouch through the discharge port.

11. A fluid injection apparatus as claimed in claim 10 wherein the pinch sleeve comprises an elastomeric sleeve shaped to receive the flexible pouch.

12. A fluid injection apparatus as claimed in claim 10 wherein the closure defines an aperture for passage of medical tubing for connection to the discharge port on the flexible pouch.

13. A fluid injection apparatus comprising:
an injector comprising an injector head and a drive piston extendable from the injector head;
an adaptor pump associated with the injector head and comprising a pressurizing body and a plunger seated in the pressurizing body;
a container body defining a internal chamber in fluid communication with the interior of the pressurizing body, the container body defining an opening allowing access to the internal chamber;
a pinch sleeve inserted in the container body opening and disposed in the internal chamber, the pinch sleeve adapted to receive a flexible pouch containing medical liquid; and
a closure closing the container body opening and enclosing the pinch sleeve in the internal chamber.

14. A fluid injection apparatus as claimed in claim 13 wherein the pressurizing body and container body are integrally formed of molded plastic material.

15. A fluid injection apparatus as claimed in claim 13 wherein the pressurizing body and container body are connected by a bridge.

16. A fluid injection apparatus as claimed in claim 15 wherein the interior of the pressurizing body and internal chamber are in fluid communication via a port defined in the bridge.

17. A fluid injection apparatus as claimed in claim 15 further comprising a pressure jacket engaged with the injector head and adapted to receive the pressurizing body to associate the pressurizing body with the injector head, and wherein the pressure jacket defines a longitudinal slot for receiving the bridge.

18. A fluid injection apparatus as claimed in claim 13 further comprising a pressure jacket engaged with the injector head and defining a receiving bore adapted to receive the pressurizing body to associate the pressurizing body with the injector head.

19. (canceled)

20. A fluid injection apparatus as claimed in claim 13 wherein the pinch sleeve comprises an elastomeric sleeve shaped to receive the flexible pouch.

21. A fluid injection apparatus as claimed in claim 13 wherein the closure defines an aperture for passage of medical tubing for connection to a discharge port on the flexible pouch.

22-23. (canceled)

24. A fluid injection apparatus as claimed in claim 13 further comprising a pouch sensor disposed in the pinch sleeve.

25-26. (canceled)

27. A method of preparing a fluid injection apparatus for a fluid injection procedure, comprising:
providing an injector comprising an injector head and a drive piston extendable from the injector head;
providing an adaptor pump comprising a pressurizing body and a plunger seated in the pressurizing body, wherein the drive piston is adapted to impart motive forces to the plunger, and wherein a container body defining an internal chamber is in fluid communication with the interior of the pressurizing body and defines an opening allowing access to the internal chamber;
associating the pressurizing body with the injector head;
inserting a pinch sleeve into the container body through the container body opening, the pinch sleeve adapted to receive a flexible pouch containing medical liquid; and
inserting a flexible pouch containing medical liquid into the pinch sleeve.

28. A method as claimed in claim 27 further comprising closing the container body opening with a closure to enclose the pinch sleeve in the internal chamber.

29. A method as claimed in claim 28 further comprising connecting medical tubing to a discharge port on the flexible pouch and passing the medical tubing and/or discharge port through an aperture defined in the closure.

30. A method as claimed in claim 27 wherein the step of associating the pressurizing body with the injector head comprises inserting the pressurizing body into a pressure jacket connected to the injector head.

31. A method as claimed in claim 27 further comprising detecting at least the presence of the pouch in the pinch sleeve with a punch sensor disposed in the pinch sleeve.
